(19) United States
(12) Patent Application Publication
(10) Pub. No.: US 2025/0032637 A1
(43) Pub. Date: Jan. 30, 2025

(54) SOLID PARTICLES COMPRISING DECELLULARIZED CARDIAC TISSUE EXTRACELLULAR MATRIX AND METHODS FOR MAKING AND USING THE SAME

(71) Applicant: Case Western Reserve University, Cleveland, OH (US)

(72) Inventors: Sam SENYO, Cleveland, OH (US); Xinming WANG, Cleveland, OH (US); Ali ANSARI, Cleveland, OH (US)

(21) Appl. No.: 18/716,576

(22) PCT Filed: Dec. 7, 2022
(86) PCT No.: PCT/US2022/052180

§ 371 Date: Jun. 5, 2024

Related U.S. Application Data
(60) Provisional application No. 63/286,593, filed on Dec. 7, 2021.

(51) Int. Cl. A61K 47/69; A61K 45/06; A61P 9/00

(52) U.S. Cl. CPC A61K 47/6901; A61K 45/06; A61K 47/6927; A61P 9/00

(57) ABSTRACT

The present disclosure relates generally to solid particles comprising decellularized cardiac tissue extracellular matrix and, more particularly, to solid particles comprising decellularized cardiac tissue extracellular matrix for use in biomolecule delivery, treatment of cardiac diseases or cardiac conditions, and methods of generating the solid particles.

### CROSS-REFERENCE TO RELATED APPLICATIONS

This application claims the benefit of U.S. Provisional Application No. 63/286,593, filed Dec. 7, 2021, entitled “SOLID PARTICLES COMPRISING DECELLULARIZED EXTRACELLULAR MATRIX AND METHODS FOR MAKING AND USING THE SAME”, which is hereby incorporated by reference in its entirety for all purposes.

### TECHNICAL FIELD

The present disclosure relates generally to solid particles comprising decellularized cardiac tissue extracellular matrix and, more particularly, to solid particles comprising decellularized cardiac tissue extracellular matrix for use in biomolecule delivery, treatment of cardiac diseases or cardiac conditions, and methods of generating the solid particles.

### BACKGROUND

Cardiovascular disease is the leading cause of death worldwide. A heart attack, or myocardial infarction (MI), is one of the major fatal cardiovascular diseases. Heart injury in adult mammals results in permanent fibrosis and compromised cardiac function. In the worst scenario, heart injury develops to heart failure which can only be cured by heart transplantation. Despite significant advances in tissue engineering, there is no regenerative therapy available for heart disease.

Decellularized extracellular matrix (dECM) derived biomaterials have been investigated as a direct therapy for heart injury. Current dECM-derived biomaterials are delivered to the heart as a liquid dECM hydrogel precursor or colloidal suspension. Liquid dECM hydrogels, however, are vulnerable to collagenase digestion and can degrade within a week. Furthermore, retention and stabilization of such liquid dECM hydrogels in the heart is unknown.

### SUMMARY

The present disclosure relates generally to solid particles comprising decellularized cardiac tissue extracellular matrix and, more particularly, to solid particles comprising decellularized cardiac tissue extracellular matrix for use in biomolecule delivery, treatment of cardiac diseases or cardiac conditions, and methods of generating the solid particles.

The present disclosure is based, at least in part, on the discovery by the inventors of an injectable solid dECM microparticle formulation derived from heart tissue that exhibits controlled sizing and extended stability in aqueous conditions. When delivered into an infarcted mouse heart, it was discovered that these solid dECM microparticles protect cardiac function, promote angiogenesis, and reduce left ventricular remodeling by sustained delivery of biomolecules. Advantageously, the developed solid dECM microparticles exhibited longer retention, higher stiffness, and slower protein release as compared to a liquid dECM hydrogel precursor. In addition, the solid dECM microparticles can be developed as a platform for biomolecule delivery. As such, solid dECM particles of the present disclosure can be developed as a modular therapy for heart injury.

Accordingly, one aspect of the present disclosure can include a solid particle comprising a decellularized cardiac tissue extracellular matrix.

Another aspect of the present disclosure can include a solid particle comprising a decellularized cardiac tissue extracellular matrix and at least one biomolecule that is chemically conjugated thereto.

Another aspect of the present disclosure can include a composition comprising a decellularized cardiac tissue extracellular matrix and a second component.

Another aspect of the present disclosure can include a composition comprising a decellularized cardiac tissue extracellular matrix, a second component, and at least one biomolecule that is chemically conjugated to the decellularized cardiac tissue extracellular matrix.

Another aspect of the present disclosure can include a pharmaceutical composition comprising a decellularized cardiac tissue extracellular matrix and a pharmaceutically acceptable carrier.

Another aspect of the present disclosure can include a pharmaceutical composition comprising a decellularized cardiac tissue extracellular matrix, at least one biomolecule that is chemically conjugated to the decellularized cardiac tissue extracellular matrix, and a pharmaceutically acceptable carrier.

Another aspect of the present disclosure can include a patch comprising a biodegradable bioscaffold seeded with a plurality of solid, cardiac tissue extracellular matrix particles.

Another aspect of the present disclosure can include a patch comprising a biodegradable bioscaffold seeded with a plurality of solid, cardiac tissue extracellular matrix particles, wherein the plurality of solid, cardiac tissue extracellular matrix particles further comprises at least one biomolecule that is chemically conjugated to the decellularized cardiac tissue extracellular matrix.

Another aspect of the present disclosure can include a solid particle consisting of decellularized cardiac tissue extracellular matrix.

Another aspect of the present disclosure can include a solid particle consisting of decellularized cardiac tissue extracellular matrix and at least one biomolecule chemically conjugated to the decellularized cardiac tissue extracellular matrix.

Another aspect of the present disclosure can include a method of treating a cardiac disease or cardiac condition in a subject, comprising administering to the subject a therapeutically effective amount of solid, cardiac tissue extracellular matrix particles, thereby treating the cardiac disease or cardiac condition.

Another aspect of the present disclosure can include a method of treating a cardiac disease or cardiac condition in a subject, comprising administering to the subject a therapeutically effective amount of solid, cardiac tissue extracellular matrix particles, wherein the solid, cardiac tissue extracellular matrix particles further comprise at least one biomolecule that is chemically conjugated to the decellularized cardiac tissue extracellular matrix, thereby treating the cardiac disease or cardiac condition.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1A is a histogram showing that electrospray parameters affect the size of solid decellularized extracellular matrix (dECM) microparticles. Solid dECM microparticle sizes were measured by microscopy. Low electrospray voltage and high flow rate generated larger particles than high voltage and low flow rate.

FIGS. 2A-B are a series of schematic illustrations showing dECM generated by electrospray and the associated experimental setup. FIG. 2A is a schematic showing solid dECM microparticle fabrication. dECM droplets were generated by electrospray and stabilized in oil with surfactant. Liquid dECM droplets were gelled on hotplate and washed in acetone to remove oil and ethanol. The final solidified dECM microparticles were stable in ethanol. The experimental setup is illustrated in FIG. 2B. Solid dECM microparticles (resuspended in PBS) were injected into the infarct area immediately after coronary artery ligation in juvenile mice. Echocardiography was measured 21 days post-surgery. After 3 days BrdU labeling, mice hearts were harvested for histological analysis.

FIGS. 3A-C show that solid dECM microparticles stimulate angiogenesis in post-myocardial infarction (MI) hearts. Vessels were evaluated by immunostaining for α-SMA and CD31. The density and area of vessels were measured to evaluate angiogenesis (FIG. 3A). Solid dECM microparticles increased small vessels density compared to MI-control (FIG. 3B). All vessels density (FIG. 3C). Solid dECM microparticles show a trend towards increased vessels density. (FIGS. 3B, 3C: n=4 per treatment, one-way ANOVA and Tukey's test. *p<0.05. All data presented as mean±SD. White arrows indicate smaller blood vessels).

FIG. 4A is a histogram of vessel areas.

FIGS. 5A-E show that dECM microparticles preserve cardiac function in 3-week post-MI hearts. Cardiac function was evaluated by echocardiography (FIG. 5A). Solid dECM microparticle treatment reduced left ventricle end diastolic diameter compared to MI-control (FIG. 5B). Both dECM hydrogel precursor and microparticles lowered left ventricle systolic diameter compared to MI-control; however, the end systolic diameter in dECM microparticles treated hearts was not significantly different from sham (FIG. 5C). Both solid dECM microparticles and dECM hydrogel precursor treated hearts showed increased (FIG. 5D) ejection fraction and (FIG. 5E) fractional shortening compared to MI-control. Solid dECM microparticles treated group, however, had a tighter distribution than dECM hydrogel precursor group. (FIGS. 5B, 5C, 5D, 5E: n=4 per treatment, one-way ANOVA and Tukey's test. *p<0.05, *p<0.01, **p<0.001, **p<0.0001. All data presented as mean±SD).

FIGS. 6A-B are a series of plots showing stroke volume and cardiac output in 3-week post-MI hearts. Stroke volume (FIG. 6A) and cardiac output (FIG. 6B) were preserved by solid dECM microparticles and dECM hydrogel precursor compared to MI-control. (FIGS. 6A, 6B: n=4 per treatment, one-way ANOVA and Tukey's test. *p<0.05, *p<0.01. All data presented as mean±SD).

FIGS. 7A-F show that solid dECM microparticles reduce fibrosis and fibroblast activation in post-MI hearts. Fibrosis was examined by Masson's Trichrome staining (FIG. 7A). Left ventricle wall thickness in the infract area was higher in solid dECM microparticles treated than MI-control (FIG. 7B). Solid dECM microparticles and dECM hydrogel precursor lowered fibrotic tissue area compared to MI-control (FIG. 7C). Fibroblast activation was examined by immunostaining for PDGFR-α and α-SMA (FIG. 7D). The density of activated fibroblasts was lowered by solid dECM microparticles treatment compared to MI-control (FIG. 7E). Solid dECM microparticles and dECM hydrogel precursor reduced fibroblast activation (FIG. 7F). (FIGS. 7B, 7C, 7E, 7F: n=4 per treatment, one-way ANOVA and Tukey's test. *p<0.05, *p<0.01, **p<0.0001. All data presented as mean±SD).

FIGS. 8A-C show fibroblast activation in vimentin positive cells. Fibroblast activation was examined by immunostaining for vimentin and α-SMA (FIG. 8A). Both solid dECM microparticles and dECM hydrogel precursor reduced fibroblast density compared to MI-control (FIG. 8B). Solid dECM microparticles and dECM hydrogel precursor reduced fibroblast activation compared to MI-control. But dECM hydrogel precursor treated hearts had a significantly higher fibroblast activation compared to sham (FIG. 8C). (FIGS. 8B, 8C: n=4 per treatment, one-way ANOVA and Tukey's test. *p<0.05, *p<0.01, **p<0.0001. All data presented as mean±SD).

FIGS. 9A-C show that solid dECM microparticles stimulate cardiomyocyte cell cycle activity in post-MI hearts. Cardiomyocyte cell cycle activity was examined by immunostaining for Ki67 expression and BrdU incorporation in TnT-positive cells (FIG. 9A). Solid dECM microparticles significantly increased the percentage of Ki67⁺ cardiomyocytes compared to MI-control (FIG. 9B). Both solid dECM microparticles and dECM hydrogel precursor promoted significant cardiomyocyte BrdU incorporation compared to MI-control (FIG. 9C). (FIGS. 9B, 9C: n=4 per treatment, one-way ANOVA and Tukey's test. *p<0.05, *p<0.01. All data presented as mean±SD).

FIGS. 10A-C show cardiomyocytes cell cycle activity in day 1 mice ventricle explants. Cardiomyocyte cell cycle activity in heart explants was examined by immunostaining for BrdU incorporation and PHH3 expression (FIG. 10A). A higher percentage of BrdU (FIG. 10B) and PHH3 positive (FIG. 10C) cardiomyocytes were observed compared to no dECM treated control. (FIGS. 10B, 10C: n=3 biological repeats with 3 technical repeats per group, one-way ANOVA and Tukey's test. *p<0.05, *p<0.01. All data presented as mean±SD).

FIGS. 11A-H show solid dECM microparticle and hydrogel characterization. Representative SEM images of dECM hydrogels (FIG. 11A) and dECM microparticles (FIG. 11B). Atomic force microscopy results indicate that dECM microparticles (4.5±0.7 kPa) have higher elastic modulus than hydrogels (2.5±0.3 kPa) (FIG. 11C). dECM hydrogels release soluble proteins in aqueous buffer within 24 h. The solid dECM microparticles exhibit a slower protein release rate (FIG. 11D). In an infinite dilution experiment over 14 days, most of the diffusible proteins were released from hydrogels within 2 days, whereas solid dECM microparticles showed a more consistent protein release rate (FIG. 11E). The dECM hydrogels and microparticles swelling and water uptake were evaluated by microscopy and analytical balance (FIG. 11F). The area of solid dECM microparticles increases by 70% within 10 min in water, dECM hydrogels showed 3% increase in dimensions after immersing in water (FIG. 11G). The weight of solid dECM microparticles increased by 95% after absorbing water, while that of dECM hydrogels increased by 85% (FIG. 11H). (FIG. 11C: n=150 to 170 measurements, unpaired t-test. FIGS. 11G, 11H: n=3 per treatment, unpaired t-test. *p<0.05, *p<0.01. All data presented as mean±SD).

FIGS. 12A-E show collagenase digestion and retention of solid dECM microparticle samples. Solid dECM microparticles and hydrogels were digested by collagenase for 24 h (FIG. 12A). dECM hydrogels were completely digested within 2 h, while the complete digestion of solid dECM microparticles took more than 24 h (FIG. 12B). Solid dECM microparticles and dECM hydrogel precursor labeled by WGA-Alexa Flour 488 were injected into porcine heart tissue explants cultured for 14 days (FIG. 12C). Marginal levels of dECM hydrogel precursor can be observed on day 14. The areas of labeled solid dECM microparticles decreased by 50% from day 1 to day 14 (FIG. 12D). The average fluorescence intensity of the remaining samples decreased by 30% in solid dECM microparticles and 60% in dECM hydrogel precursor (FIG. 12E). (FIG. 12B: 3 repeats per group. FIGS. 12D, 12E: 3 repeats per group, unpaired t-test. *p<0.05, *p<0.01. All data presented as mean±SD. FIG. 12A: dash line circles dECM).

FIGS. 13A-H show that solid dECM microparticles provide platform technology for drug loading and tissue targeting. Size distribution of original electrospray dECM microparticles (22.7±13.7 μm) (FIG. 13A). Size distribution of sonicated microparticles (3.4±2.4 μm) (FIG. 13B). Size distribution of bead-milled microparticles (2.3±0.6 μm), all by microscopy (FIG. 13C). Polystyrene beads were incorporated into dECM microparticles (FIG. 13D). Dextran was also incorporated into solid dECM microparticles by electrospray of pre-mixed dECM and dextran solution (FIG. 13E). A controlled release of dextran was observed from solid dECM microparticles (FIG. 13F). NHS-AF488 was used for solid dECM microparticle coating. A brighter fluorescent signal was observed in NHS-AF488 functionalized particles than passively adsorbed control (FIG. 13G). FTIR of NHS-AF488 treated samples. Increased C—N stretching and C—H bending were observed in NHS-AF488 modified microparticles (FIG. 13H). (FIG. 13F: n=3 technical repeats per condition, no statistical comparison applied. All data presented as mean±SD).

### DETAILED DESCRIPTION

### Definitions

Unless defined otherwise, all technical and scientific terms used herein have the same meaning as is commonly understood by one of skill in the art to which the present disclosure pertains.

Throughout the description and claims of this specification, the word “comprise” and variations of the word, such as “comprising” and “comprises,” means “Including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

In the context of the present disclosure, the term “about”, when expressed as from “about” one particular value and/or “about” another particular value, also specifically contemplated and disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these aspects are explicitly disclosed.

Optionally, in some aspects, when values or characteristics are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

As used herein, phrases such as “between X and Y” and “between about X and Y” can be interpreted to include X and Y.

As used herein, phrases such as “between about X and Y” can mean “between about X and about Y”.

As used herein, phrases such as “from about X to Y” can mean “from about X to about Y”.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms can encompass different orientations of the device in use or operation in addition to the orientation depicted in the figures. For example, if the device in the figures is inverted, elements described as “under” or “beneath” other elements or features would then be oriented “over” the other elements or features.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the terms “optionally” and “optional” can mean that the subsequently described event, circumstance, or material may or may not occur or be present, and that the description includes instances where the event, circumstance, or material occurs or is present and instances where it does not occur or is not present.

As used herein, the term “hydrogel” can refer to a cross-linked network of water-soluble polymers that swells in water to form a stable gel material.

As used herein, the term “hydrogel precursor” can refer to natural mammalian tissue depleted of cells (i.e., decellularized) by surfactants and then digested by proteases to form a viscous protein suspension.

As used herein, the term “extracellular matrix” or “ECM” can refer to a complex network of materials produced and secreted by the cells of the tissue into the surrounding extracellular space and/or medium and which typically together with the cells of the tissue impart the tissue its mechanical and structural properties. Generally, the ECM includes fibrous elements (particularly collagen, elastin, and/or reticulin), cell adhesion polypeptides (e.g., fibronectin, laminin and/or adhesive glycoproteins), and space-filling molecules (usually glycosaminoglycans (GAG), proteoglycans).

As used herein, the term “cardiac tissue”, when referring to the source from which solid dECM particles of the present disclosure are derived or made, can refer to contractile tissue including atria, ventricles, and septum of the heart and associated subdomains including endocardium, epicardium, and myocardium.

As used herein, the terms “decellularized extracellular matrix” or “dECM” can refer to the extracellular matrix that supports organization of a particular tissue (e.g., cardiac tissue), which has undergone a decellularization process (e.g., a removal of all cells from the tissue) and is thus devoid of cellular components but retains the structural properties of the ECM from which the dECM is derived or obtained.

As used herein, the term “cardiac tissue regeneration” can refer to repair of damaged or diseased cardiac tissue by protection of stressed cardiac cells, generation of cardiac cells and networks including cardiomyocytes, vasculature, nerves, and lymphatic vessels by proliferation, differentiation or transplantation, protection from or reversal of adverse tissue remodeling of wall geometry (i.e., fibrosis, cardiomyocyte hypertrophy), acute and chronic inflammation, and removal of necrotic tissue. These phenomena of cellular and tissue repair consistent with cardiac regeneration lead to protection of, or recovery to, life-sustainable cardiac function as assessed, for example, by contractile and hemo-dynamics.

As used herein, the term “cellular components” can refer to cell membrane components or intracellular components which make up a cell. Examples of cell components include cell structures (e.g., organelles) or molecules comprised of the same. Examples of such include, but are not limited to, cell nuclei, nucleic acids, residual nucleic acids (e.g., fragmented nucleic acid sequences), cell membranes and/or residual cell membranes (e.g., fragmented membranes) that are present in cells of a particular tissue. It will be appreciated that due to the removal of all cellular components from a tissue, a dECM derived therefrom cannot induce an immunological response when implanted in a subject.

As used herein, the term “devoid of cellular components” can refer to being more than 90%, 91%, 92%, 93%, 94%, 95%, 96%, 97%, 98%, or 99%, (e.g., 100%) devoid of the cellular components present in the natural (e.g., native) tissue (e.g., cardiac tissue) from which ECM is derived.

As used herein, the term “pharmaceutical composition” can refer to a preparation of one or more of the solid dECM particles described herein, with other chemical components such as pharmaceutically suitable carriers and excipients. The purpose of a pharmaceutical composition is to facilitate administration of the solid dECM particles to a subject.

As used herein, the term “pharmaceutically acceptable carrier” can refer to a carrier or a diluent that does not cause significant irritation to a subject and does not abrogate the biological activity and properties of the administered agent (e.g., solid dECM particle(s)). Examples, without limitations, of carriers are propylene glycol, saline, emulsions and mixtures of organic solvents with water.

As used herein, the term “excipient” can refer to an inert substance added to a pharmaceutical composition to further facilitate administration of an agent (e.g., solid dECM particle(s)). Examples, without limitation, of excipients include calcium carbonate, calcium phosphate, various sugars and types of starch, cellulose derivatives, gelatin, vegetable oils and polyethylene glycols.

As used herein, the term “solid”, as it is used with reference to the dECM particles of the present disclosure, can refer to particles that have a three-dimensional shape and, ex vivo, are not a liquid, sol-gel (e.g., a hydrogel), or a gas at ambient temperatures (e.g., room temperature).

As used herein, the term “cardiac tissue” can refer to any part of the heart including the pericardium, the endocardium, the myocardium and the epicardium, as well as blood vessels connected to the heart.

As used herein, the term “cardiac tissue injury” can refer to a cardiac tissue that is, for example, ischemic, infarcted, reperfused, or otherwise focally or diffusely injured or diseased. Injuries associated with a cardiac tissue include any areas of abnormal tissue in the heart, including any areas of abnormal tissue caused by a disease, disorder or injury and includes damage to the epicardium, endocardium and/or myocardium. Non-limiting examples of causes of cardiac tissue injuries include acute or chronic stress (e.g., systemic hypertension, pulmonary hypertension or valve dysfunction), atheromatous disorders of blood vessels (e.g., coronary artery disease), ischemia, infarction, inflammatory disease and cardiomyopathies, myocarditis or congestive heart failure.

As used herein, the term “cardiomyocytes” can refer to fully or at least partially differentiated cardiomyocytes.

As used herein, the term “biomolecule” can refer to organic and inorganic molecules (e.g., macromolecules or small molecules), such as proteins, enzymes, antibodies, nucleic acids (e.g., DNA, siRNA), drugs, and pharmaceutical compositions comprising one or more drugs. In some instances, a biomolecule can include a non-native biomolecule. In some instances, a biomolecule can include a native biomolecule; that is, a biomolecule that originates from, or is native to, a cardiac tissue source from which a solid dECM particle of the present disclosure is derived or made. For example, a native biomolecule can include a component (e.g., proteoglycan, collagen, elastin, fibronectin, laminin) that naturally forms or comprises part of a cardiac tissue ECM.

Non-limiting examples of native or non-native biomolecules (e.g., that modify cellular activity) that can comprise the solid dECM particles of the present disclosure (e.g., which can be chemically conjugated, e.g., encapsulated in, attached to, coated on, embedded or impregnated)) include, but are not limited to, agrin, nerve growth factor (NGF), interleukin 4, interleukin 10, bone morphogenetic protein-2 (BMP-2), vascular endothelial growth factor (VEGF), angioprotein (Ang-1), fibroblast growth factor, (FGF-2), Insulin-like growth factor (IGF-1), stromal cell derived factor-1 (SDF-1), platelet derived growth factor (PDGF), Periostin, Neuregulin, Follistatin-like 1 (FSTL1), Fibronectin splice variants (Fibronectin EDB), and Versican.

As used herein, the term “non-native”, when referring to a biomolecule, can mean a biomolecule that does not originate from, or is not native to, a cardiac tissue source from which a solid dECM particle of the present disclosure is derived or made. For example, a “non-native biomolecule” can include a biomolecule that has been made in vitro. In some instances, a “non-native biomolecule” can include a synthetic, artificial, or non-naturally occurring biomolecule. A synthetic biomolecule can include a non-naturally occurring biomolecule that arises from human engineering and which is produced, e.g., in vitro, in vivo (i.e., in a living organism), or produced in vivo and subsequently isolated and purified.

As used herein, the term “chemically conjugated”, when referring to an association between a solid dECM particle of the present disclosure and a biomolecule, can mean attached through a chemical bond, such as an ionic bond, a covalent bond, hydrogen bond (including van der Waals forces), metallic bond, or a combination thereof.

As used herein, the term “macromolecule” can refer to a large organic molecule. Examples of macromolecules can include, but are not limited to, nucleic acids, oligonucleotides, proteins, peptides, carbohydrates, polysaccharides, glycoproteins, lipids, hormones, drugs, and prodrugs.

As used herein, the term “small molecule” can refer to a molecule that has a molecular weight of less than about 1500 g/mol. A small molecule can be, for example, small organic molecules, peptides or peptide-like molecules.

As used herein, the term “drug” can refer to a wide spectrum of agents that can be used for human applications including, but not limited to, treatment and study. The term broadly includes proteins, peptides, hormones, vaccines and small molecules.

As used herein, the terms “cardiac disease” or “cardiac condition” can refer to any alteration of heart function based upon parameters including, but not limited to, electrical and/or muscular balance. Specific cardiac diseases may include, but are not limited to, MI, ischemic cardiomyopathy, angina pectoris, heart rhythm arrhythmias, tachycardias, congestive heart failure, and/or atrial fibrillation, nerve conduction disorders, thrombophilia, atherosclerosis, hypertension, arteriosclerosis, cardiomyopathy, hypertension, and/or arterial or venous stenosis, or valvular heart disease.

As used herein, the term “subject” can refer to a vertebrate, such as a mammal (e.g., a human). Mammals can include, but are not limited to, humans, dogs, cats, horses, cows, and pigs.

As used herein, the term “therapeutically effective amount” can refer to an amount sufficient to affect a beneficial or desired clinical result, which dose could be administered in one or more administrations. According to one embodiment, a single administration is employed. The injection can be administered into any site in which cardiac tissue regeneration is required. For example, for treatment of a cardiac tissue injury, solid dECM particles of the present disclosure can be administered into various regions of the heart (e.g., into an infarct area) depending on the type of cardiac tissue repair required. Intramyocardial administration is particularly advantageous for repairing cardiac tissue in a subject having a cardiac disorder characterized by cardiac arrhythmia, impaired, cardiac conducting tissue or myocardial ischemia.

As used herein, the term “treating” can include abrogating, substantially inhibiting, slowing, or reversing the progression of a cardiac disease or cardiac condition, substantially ameliorating clinical or aesthetical symptoms of a cardiac disease or cardiac condition, or substantially preventing the appearance of clinical or aesthetical symptoms of a cardiac disease or cardiac condition.

As used herein, the term “isolated”, when referring to solid dECM particles of the present disclosure, can refer to solid dECM particles that are not associated with (e.g., physically removed or separate from) the in vivo environment, such as cells, cellular components, blood, etc.

### Overview

The present disclosure is based, at least in part, on the surprising discovery that solid dECM microparticles derived from cardiac tissue and generated by electrospray achieved sustained release of diffusible biomolecules while being retained in cardiac tissue for at least two weeks, thereby improving cardiac post-injury response in non-regenerative hearts. It was also surprisingly discovered that the solid dECM microparticles induced robust angiogenesis and reduced ventricle remodeling. Finally, the inventors of the present disclosure demonstrated that the solid dECM microparticles can advantageously provide a platform for delivery of biomolecules in vivo, such as macromolecules and/or drugs.

Based at least on the foregoing discoveries, the present disclosure generally provides solid particles comprising dECM and methods of generating the solid dECM particles and, more particularly, solid dECM particles that are derived from cardiac tissue and which can be used as a platform for biomolecule delivery.

Solid dECM Particles

One aspect of the present disclosure can include an isolated, solid dECM particle, or isolated solid dECM particles, derived or made from cardiac tissue. Solid dECM particles of the present disclosure contain ECM structural proteins like collagen I, fibronectin, laminin and fibrillin, in addition to growth factors that direct proliferation and differentiation, such as agrin and periostin, and angiogenesis factors, such as heparan sulfate proteoglycan 2 and alpha(B)-crystallin. As such, the proteome of the solid dECM particles advantageously has sensitivity to proteases in certain areas of a cardiac tissue injury (e.g., an infarct area), which can influence cardiac cell phenotypes by macromolecules released by passive diffusion and degradation. As discussed below, solid dECM particles of the present disclosure advantageously exhibiting one or more of the following characteristics as compared to a liquid dECM hydrogel precursor: (1) longer retention in a target cardiac tissue; (2) higher stiffness; (3) slower protein release in vivo; and (4) extended stability in aqueous conditions.

In one embodiment, solid dECM particles of the present disclosure can be composed or made of about 10% (e.g., more than 10%), 20%, 30%, 40%, 50%, 60%, 70%, 80%, 90%, 95%, or 99% (e.g., 100%) by mass (g or mg) of dECM derived from cardiac tissue. In one example, solid dECM particles of the present disclosure can be composed or made entirely (100%) of dECM derived from cardiac tissue.

In another embodiment, solid dECM particles of the present disclosure can be derived from autologous or non-autologous cardiac tissue. In some instances, the cardiac tissue from which the solid dECM particles are derived is a human or non-human mammal. In one example, solid dECM particles of the present disclosure are derived from a porcine ventricle.

In another embodiment, the solid dECM particles are devoid of cellular components.

In some instances, solid dECM particles of the present disclosure are distinct spheres of a homogeneous size. In such instances, the solid dECM particles can have a regular shape such that they are capable of being injected without sticking to one another in a syringe or catheter. In other instances, solid dECM particles of the present disclosure can have an oval, an irregular, or any other shape.

In another embodiment, solid dECM particles of the present disclosure are microparticles having a diameter, in the longest dimension, of, e.g., between about 1-300 μm, about 2-200 μM, or about 3-100 μM. In one example, solid dECM particles of the present disclosure are microparticles between about 1-100 μm. In another example, solid dECM particles of the present disclosure are microparticles having a diameter, in the longest dimension, of about 20 μm.

In another embodiment, solid dECM particles of the present disclosure are nanoparticles having a diameter, in the longest dimension, of, e.g., about 1-1000 nm, about 10-1000 nm, or about 100-1000 nm.

In another embodiment, solid dECM particles of the present disclosure have an elastic modulus that is greater than an elastic modulus of a hydrogel or hydrogel precursor. For example, solid dECM particles of the present disclosure can have an elastic modulus of about 1.5 kPa to about 6.5 kPa, about 3 kPa to about 5 kPa, or about 4.5 kPa. In another example, solid dECM particles of the present disclosure have an elastic modulus of greater than about 2.5 kPa, about 3 kPa, about 3.5 kPa, about 4 kPa, about 4.5 kPa (e.g., 4.5 kPa), about 5 kPa, about 5.5 kPa, about 6 kPa, about 6.5 kPa, or greater than about 2.5 kPa but less than about 6.5 kPa. It will be appreciated that the elastic modulus of solid dECM particles of the present disclosure can be increased by increasing the density (e.g., concentration of proteins) of the solid dECM particles using, for example, electrocompaction (see Wang X. et al., “Injectable Extracellular Matrix Microparticles Promote Heart Regeneration in Mice with Post-ischemic Heart Injury,” Adv Healthc Mater. 2022 April; 11(8):e2102265).

In another embodiment, solid dECM particles of the present disclosure, when exposed to or immersed in an aqueous environment (e.g., a bodily fluid, such as blood), can swell up to about 50-90%, about 60-80%, or about 70% as compared to dECM particles or hydrogel in a non-aqueous environment. The ability of the solid dECM particles to swell when exposed to or immersed in an aqueous environment can be referred to as “the swellability of solid dECM particles” and can refer to the amount of water absorption of the solid dECM particles from a dry state to an aqueous state. The degree of swellabililty can be assessed using known techniques. In one example, solid dECM particle swelling and water uptake can be evaluated by microscopy and analytical balance by, e.g., measuring an initial diameter of the particles in a dry state and then measuring the diameter of the particles after exposure to a fluid (e.g., water) after a period of time (e.g., 10 minutes). The area of the particles at each measurement time can then be calculated and compared. In another example, the weight (in g or μg) of solid dECM particles before and after exposure to a fluid can be compared.

In another embodiment, solid dECM particles of the present disclosure, in a physiological environment (e.g., inside the body of a subject), can release native or non-native biomolecules, at a controlled rate over a period of time, which can be assayed using the infinite dilution (or protein release) assay as described in the Example below and by Wang X. et al., “Injectable Extracellular Matrix Microparticles Promote Heart Regeneration in Mice with Post-ischemic Heart Injury,” Adv Healthc Mater. 2022 April; 11(8):e2102265. In some instances, the period of time is about 2 hours to about 48 hours, about 2-8 hours, about 8-12 hours, about 12-16 hours, about 16-20 hours, about 20-24 hours (e.g., about 24 hours), about 24-28 hours, about 28-32 hours, about 32-36 hours, about 36-40 hours, about 40-44 hours, or about 44-48 hours.

The term “controlled release rate” can refer to an attribute or characteristic of solid dECM particles of the present disclosure indicating that a native or non-native biomolecule is released into a physiological environment (e.g., inside the body of a subject) in a controlled fashion, rather than immediately. Thus, a “controlled release rate” can include no more than about 20-50% of total released biomolecule within about 20-50 hour(s) when the solid dECM particles are released to a physiological environment (e.g., inside the body of a subject). In one example, a controlled release rate can include no more than about 20%, no more than about 25%, no more than about 30%, no more than about 35%, no more than about 40% (e.g., no more than about 37%), no more than about 45%, or no more than about 50% of total released biomolecule within a physiological environment within about 20 hours, about 25 hours, about 30 hours, about 35 hours, about 40 hours, about 45 hours, or about 50 hours (e.g., about 48 hours). In a further example, a controlled release rate can include no more than about 37% of total released biomolecule within a physiological environment within about 48 hours.

In one aspect, “controlled release” excludes “immediate release” formulations, such as with the burst release of proteins observed in hydrogel precursors (see Example below) in which soluble proteins loosely bound to or associated with the hydrogel precursors are released rapidly in an aqueous environment. In one example, solid dECM particles of the present disclosure exhibit a slower biomolecule (e.g., protein) release rate as compared to a biomolecule (e.g., protein) release rate of a hydrogel or hydrogel precursor over a period of time (as discussed above).

In one example, solid dECM particles of the present disclosure can exhibit a controlled release rate (measured, e.g., as parts per million, ppm, of biomolecule mass (e.g., protein mass) over time) of about 0.5 ppm/hour to about 4 ppm/hour, or about 0.5-1 ppm/hour, or about 1-1.5 ppm/hour, or about 1.5-2 ppm/hour (e.g., 2 ppm/hour), or about 2-2.5 ppm/hour, or about 2.5-3 ppm/hour, or about 3-3.5 ppm/hour, or about 3.5-4 ppm/hour, or about 4-4.5 ppm/hour, or about 4.5-5 ppm/hour. In another example, solid dECM particles of the present disclosure can exhibit a controlled release rate of biomolecule mass (e.g., protein mass) of about 35 ppm over 24 hours to about 45 ppm over 24 hours, or about 35-36 ppm over 24 hours, or about 36-37 ppm over 24 hours, or about 37-38 ppm over 24 hours (e.g., about 38 ppm over 24 hours), or about 38-39 ppm over 24 hours, or about 39-40 ppm over 24 hours, or about 40-41 ppm over 24 hours, or about 41-42 ppm over 24 hours, or about 42-43 ppm over 24 hours, or about 43-44 ppm over 24 hours, or about 44-45 ppm over 24 hours.

Retention of solid dECM particles at an injection or administration site of a subject concentrates the solid dECM particles in a defined region despite increased fenestration of tissue associated with chronic disease and ischemic damage. Unlike the solid dECM particles of the present disclosure, dECM hydrogels and hydrogel precursors, when injected into the heart, spread as a liquid within the beating tissue preceding the gelation that takes several minutes, which reduces retention at the injection site. It was surprisingly discovered that the solid nature of the dECM particles of present disclosure advantageously facilitates a controlled release rate of biomolecules (e.g., proteins) at repeatable rates, thereby promoting controlled dosing of treatment using the solid dECM particles.

In another embodiment, solid dECM particles of the present disclosure are not a liquid or sol-gel (e.g., hydrogel) ex vivo. In one example, solid dECM particles of the present disclosure are not a liquid or sol-gel (e.g., hydrogel) ex vivo at room temperature (e.g., about 68 to 77° F. (20 to 25° C.)).

In another embodiment, solid dECM particles can gel at physiological temperature (37° C.). Gelation can be accelerated by higher temperatures (e.g., 50° C.). Alternatively, gelation can be induced by freezing (e.g., at about −20° C.) before returning to a solid at room temperature.

In another embodiment, solid dECM particles of the present disclosure exhibit increased resistance to degradation (e.g., by collagenase) as compared to a hydrogel. In one example, solid dECM particles of the present disclosure can resist enzymatic degradation (e.g., complete degradation by collagenase) about 1.5 times, about 2 times, about 2.5 times, about 3 times, about 3.5 times, about 4 times, about 4.5 times, about 5 times, about 5.5 times, about 6 times, about 6.5 times, about 7 times, about 7.5 times, about 8 times, about 8.5 times, about 9 times, about 9.5 times, about 10 times, about 10.5 times, or about 11 times greater than a degradation time (e.g., complete degradation by collagenase) for a hydrogel. In a further example, solid dECM particles of the present disclosure do not exhibit complete degradation by collagenase for about 24 hours.

In another aspect, solid dECM particles of the present disclosure can serve as a platform for delivery of one or more native or non-native biomolecules to a target site (e.g., a tissue in need of repair or regeneration, such as a diseased or dysfunctional cardiac tissue) in vivo. Methods for preparing the solid dECM particles as a platform for in vivo biomolecule delivery are discussed below and include, but are not limited to, direct loading of native or non-native biomolecules into solid dECM particles, solid dECM particle surface functionalization, and incorporating biomolecule-loaded particles into or onto solid dECM particles of the present disclosure.

In one embodiment, one or more non-native biomolecules can be chemically conjugated with, or to, solid dECM particles of the present disclosure by direct loading. Techniques for directly loading biomolecules (e.g., growth factors and hydrophilic small molecules) into and/or onto dECM particles are known in the art and include, for example, mixing and emulsion whereby the heterogenous composition of proteins across a range of isoelectric values allows for incorporation of a range of hydrophobic and hydrophilic drugs. Non-limiting examples of such techniques for direct loading of biomolecules into and/or onto solid dECM particles, any one or combination of which may be used to directly load biomolecules onto and/or into solid dECM particles of the present disclosure, are described by S. Bheri and M. E. Davis, “Nanoparticle-Hydrogel System for Post-myocardial Infarction Delivery of MicroRNA,” ACS Nano, Vol. 13, No. 9., American Chemical Society, pp. 9702-9706 (September-2019), C. Fan et al., “Nanoparticle-Mediated Drug Delivery for Treatment of Ischemic Heart Disease,” Frontiers in Bioengineering and Biotechnology, Vol. 8. Frontiers Media S.A. (June-2020), and Wang X. et al., “Injectable Extracellular Matrix Microparticles Promote Heart Regeneration in Mice with Post-ischemic Heart Injury,” Adv Healthc Mater. 2022 April; 11(8):e2102265.

In another embodiment, one or more non-native biomolecules can be chemically conjugated with, or to, solid dECM particles of the present disclosure by functionalizing all or only a portion of a surface comprising the solid dECM particles. Such surface functionalization can include any suitable surface functionalization, such as for improving colloidal stability, for obtaining a certain surface charge, for coupling of pro-angiogenic agents or growth factors, or for targeting a particular analyte (e.g., a cell surface receptor). In some instances, at least one surface of a solid dECM particle is functionalized to include one or a combination of chemical and/or biological moieties, such as PEI, PEG, polysaccharides, reactive groups (e.g., amino, carboxyl, tosyl, aldehyde, epoxy, hydrazide and others), drugs, lectins, antibodies, proteins (e.g., streptavidin or avidin), peptides, or nucleic acids. In one example, one or more chemical agents can be used to modify amino acid side chains on protein or peptides of solid dECM particles, including established amine-amine, sulfhydryl-to-sulfhydryl to conjugates synthetic molecules to the microparticle surface compared to passive adsorption. Examples includes NHS ester and NHS-maleimide, as described in the Example below.

In another embodiment, one or more non-native biomolecules can be chemically conjugated with, or to, solid dECM particles of the present disclosure by loading one or more biomolecule-loaded particle onto and/or into the solid dECM particles. In one example, one or more non-native biomolecule can be loaded onto and/or into a particle, such as a polymeric particle (e.g., a polymeric bead comprising PEI, PEG, a polysaccharide, etc.), which is then loaded into and/or onto the solid dECM particles of the present disclosure. Techniques for doing so are known in the art. Generally, direct mixing allows for encapsulation of biomolecules (e.g., cells, smaller particles, and drugs) using an emulsion by direct agitation (sonication, vortexing) and fluidics-based mixing (double emulsion). The sonication approach provides high yield and must be followed by filtration to account for wide polydispersity. A microfluidics or electrospray based double emulsion provides greater control of doping ratios. Non-limiting examples of such techniques include those described by Wang X. et al., “Injectable Extracellular Matrix Microparticles Promote Heart Regeneration in Mice with Post-ischemic Heart Injury,” Adv Healthc Mater. 2022 April; 11(8):e2102265, and Marinelli L. et al., “Preparation, Characterization, and Biological Evaluation of a Hydrophilic Peptide Loaded on PEG-PLGA Nanoparticles”, Pharmaceutics, 14(9):1821 (August 2022).

As well as, or instead of, the biomolecules described herein above, another aspect of the present disclosure can include incorporating (e.g., encapsulating) cells into the solid dECM particles of the present disclosure. By “encapsulates” or “encapsulate”, it is meant that cells are located within the solid dECM particle(s); that is, the cells are not seeded or present on the outside of the solid dECM particle(s), but rather are contained within the boundaries of the solid dECM particle(s). The cells may be derived from any organism including, for example, mammalian cells (e.g., human). In one example, the cells can comprise stem cells, e.g., adult stem cells, such as mesenchymal stem cells or pluripotent stem cells, such as embryonic stem cells or induced pluripotent stem cells. The stem cells may be modified so as to undergo ex vivo differentiation. The cells are preferably intact (i.e., whole) and preferably viable; although, it will be appreciated that pre-treatment of cells, such as generation of cell extracts or non-intact cells are also contemplated by the present disclosure.

In one example, the cells are cardiac cells (e.g., human cardiomyocytes). Cardiomyocytes may be derived from cardiac tissue or from stem cells (such as embryonic stem cells or adult stem cells, such as mesenchymal stem cells). Methods of differentiating stem cells along a cardiac lineage are known in the art. See, for example, Muller-Ehmsen J., et al., Circulation 2002; 105:1720-6; Zhang M, et al., J Mol Cell Cardiol. 2001: 33:907-21; Xu et al., Circ Res. 2002; 91:501-508; and U.S. Pat. Appl. Pub. No. 2005/0037489. In one example, the cardiomyocytes are Ki67.

### Devices

Another aspect of the present disclosure can include a seeded patch for attachment (e.g., epicardial attachment) to or over a myocardial region including an injured area to control post-injury remodeling of the myocardial region. Advantageously, use of a seeded patch of the present disclosure can result in enhanced transient mechanical support for an injured myocardial region and/or a region surrounding the injured myocardial region, enhanced angiogenesis, enhanced localization of stem cells, and/or reduced adverse effects, e.g., ventricular remodeling, including reduced fibrosis, of the heart, thereby leading to enhanced function of an infarcted region.

In one embodiment, a seeded patch can comprise a biodegradable bioscaffold seeded with a plurality of solid dECM particles of the present disclosure. In one example, the bioscaffold can comprise one or a combination of biodegradable polymers, such as natural or synthetic polymers. Non-limiting examples of natural polymers can include polysaccharide polymeric materials (e.g., chitosan, chitin, starch, and cellulose), hyaluronic acid, poly(hydroxybutyrate), poly(hydroxyvalerate), polyhydroxyhexanoate, and poly(hydroxyalkanoates) (PHAs). Non-limiting examples of synthetic polymers include PLA, polycaprolactone, polybutylene succinate, polybutylene succinate adipate, aliphatic-aromatic copolyesters, polybutylene adipate/terephthalate, and polymethylene adipate/terephthalate.

A seeded patch of the present disclosure can have any shape and dimensions desired to accommodate the contour of a particular myocardial region including an injured area. As such, a seeded patch can have a regular (e.g., rectangular, square, circular) or irregular shape and comprise one or multiple layers (e.g., multi-laminate constructs can be formed by overlapping individual strips, each comprising a seeded patch, and applying pressure to the overlapped portions to fuse the strips together of a desired thickness). In one example, a seeded patch of the present disclosure can be an epicardial patch.

In one embodiment, a seeded patch comprises only (consists of) a biodegradable bioscaffold seeded with a plurality of solid dECM particles of the present disclosure.

In another embodiment, a seeded patch of the present disclosure can be applied to (contacted with) and/or adhered to (fixed to) an implantable medical device and/or other bioscaffold(s) and/or other materials with anchoring projections (such as plastic or metal pins or sutures), adhesives, or other fixation devices known to those skilled in the art. In one embodiment, a seeded patch of the present disclosure can be sutured or otherwise secured to a medical device. For example, a seeded patch can be wrapped around the medical device and redundant tissue gathered and secured via sutures. Tissue segments or sheets can be attached to each other before or during attachment to a device using surgically acceptable techniques, e.g., suturing, gluing, stapling or compressing.

Methods for Generating Solid dECM Particles

In another aspect, methods are provided for generating the solid dECM particles of the present disclosure.

In one embodiment, described in the Example below, solid dECM particles of the present disclosure can be generated by electrospray of solubilized dECM. Particle size can be controlled by tuning electrospray voltage and syringe pump flow rate.

In another embodiment, solid dECM particles of the present disclosure can be generated using a dual-input microfluidic chip whereby a pressurized flow controller drives an emulsion of dECM hydrogel precursor into emulsion with an oil-based carrier (e.g., mineral oil, fluorinated oil, such as 2 wt % 008-Fluorosurfactant in HFE7500). Microparticle size can be adjusted by flow speed, surfactant concentration, chip dimensions, and degree of dECM digestion.

It will be appreciated that techniques, other than those described herein, for incorporating cells and/or biomolecules into or onto the solid dECM particles are known in the art. For example, one way to encapsulate cells/biomolecules is using 3D printing. Inkjet bio-printing is a “noncontact” technique that uses electromagnetic technology to deposit tiny droplets of “ink” onto a substrate. Droplet size can be varied by adjusting pulse frequency and ink viscosity. Advantages of 3D printing are high reproducibility and precise control of droplet size and dose.

### Methods of Treatment

Another aspect of the present disclosure can include a method of treating a cardiac disease or cardiac condition in a subject (e.g., a human subject). One step of the method can comprise administering to the subject a therapeutically effective amount of one or more solid dECM particles of the present disclosure, thereby treating the cardiac disease or cardiac condition.

In one embodiment, the method can include treating a cardiac disease or disorder associated with a defective or absent myocardium in a subject, the method comprising transplanting a therapeutically effective amount of the solid dECM particles of the present disclosure into the subject, thereby treating the cardiac disorder associated with a defective or absent myocardium.

The method can be applied to a subject having suffered a cardiac tissue injury so as to repair cardiac tissue associated with the injury. The method can also be applied to repair cardiac tissue susceptible to, or be associated with, future onset or development of a cardiac disorder so as to inhibit such onset or development.

The present disclosure can be advantageously used to treat cardiac disorders associated with, for example, necrotic, apoptotic, damaged, dysfunctional or morphologically abnormal myocardium. Such cardiac disorders include, but are not limited to, ischemic heart disease, cardiac infarction, rheumatic heart disease, endocarditis, autoimmune cardiac disease, valvular heart disease, congenital heart disorders, cardiac rhythm disorders, impaired myocardial conductivity and cardiac insufficiency. Since the majority of cardiac diseases involve necrotic, apoptotic, damaged, dysfunctional or morphologically abnormal myocardium, and since the solid dECM particles of the present disclosure can have one or more of the following effects, (1) preservation of cardiac function, (2) reduction in fibrosis and fibroblast activation, (3) stimulation of cardiomyocyte cell cycle activity, (4) promoting angiogenesis in cardiac tissue, and (5) protection against ventricular wall remodeling. The method of repairing cardiac tissue of the present disclosure can be used to treat the majority of instances of cardiac diseases and disorders.

The phrase “preservation of cardiac function”, can refer to the absence or limited loss of cardiac function with therapy using the solid dECM particles of the present disclosure, versus without therapy, in functional cardiac metrics (e.g., ejection fraction, fractional shortening, systolic and diastolic geometry, and hemodynamics) such that cardiac function is quantitatively closer to pre-injury and/or healthy levels of cardiac function.

The phrase “reduction in fibrosis and fibroblast activation” can refer to the disease manifestation of cardiac remodeling associated with proliferation and activation of fibroblasts and immune cells, increased digestive enzyme activity, and the increased deposition of extracellular insoluble proteins, such as collagen to produce tissue with reduced cyclic contractile activity and material consistent with scar, which is/are reduced or eliminated by treatment using the solid dECM particles of the present disclosure.

By “stimulation of cardiomyocyte cell cycle activity”, it is meant that mononucleated diploid cardiomyocytes duplicated the genome for cytokinesis and generation of new cardiomyocyte daughter cells and adjacent progenitor cells duplication and differentiation into new cardiomyocytes cells as a consequence of treatment with the solid dECM particles of the present disclosure.

By “promoting angiogenesis in cardiac tissue”, it is meant that vascular cell generation of new blood vessels de novo and branching of new vessels from existing vasculature occurs as a result of treatment with the solid dECM particles of the present disclosure.

By “protection against ventricular wall remodeling”, it is meant that there is reduced inflammatory induced digestive enzymes, such as matrix metalloproteinases that digest and facilitate structural changes to the myocardial wall geometry as a result of treatment with the solid dECM particles of the present disclosure (as compared to a control level).

According to one embodiment, the method of the present disclosure can be advantageously used to efficiently reverse, inhibit, or prevent cardiac damage caused by ischemia resulting from myocardial infarction.

According to one embodiment, single or multiple administrations of a therapeutically effective amount of solid dECM particles can be employed. An injection, for example, can be administered into any site in which tissue regeneration is required. For example, for treatment of cardiac disorders, the solid dECM particles can be directly administered into various regions of the heart, depending on the type of cardiac tissue repair required. Intramyocardial administration is particularly advantageous for repairing cardiac tissue in a subject having a cardiac disorder characterized by cardiac arrhythmia, impaired, cardiac conducting tissue or myocardial ischemia. Such transplantation directly into cardiac tissue ensures that the administered cells/tissues will not be lost due to the contracting movements of the heart.

The solid dECM particles of the present disclosure can be transplanted via transendocardial or transepicardial injection, depending on the type of cardiac tissue repair being affected, and the physiological context in which the cardiac repair is affected. This allows the administered cells or tissues to penetrate the protective layers surrounding the membrane of the myocardium.

In some instances, a catheter-based approach is used to deliver a transendocardial injection. The use of a catheter precludes more invasive methods of delivery wherein the opening of the chest cavity would be necessitated.

To repair cardiac tissue damaged by ischemia, for example, due to a cardiac infarct, the solid dECM particles of the present disclosure can be administered directly into the infarct area and/or directly to the border area of the infarct. As one skilled in the art would be aware, the infarcted area is grossly visible, allowing such specific localization of application of solid dECM particles to be possible. The precise determination of an effective dose in this particular case may depend, for example, on the size of an infarct, and the time elapsed following onset of myocardial ischemia.

In another embodiment, solid dECM can be prepared as nanoparticles and then formulated, in a therapeutically effective amount, as a pharmaceutical composition, which can then be administered (e.g., via intravenous injection) into a subject suffering from, or at risk of suffering from, a cardiac disease or cardiac condition.

In any of the methods described herein, the solid dECM particles of the present disclosure can be administered either per se or, as a part of a pharmaceutical composition that further comprises a pharmaceutically acceptable carrier.

In some instances, the pharmaceutical carrier is an aqueous solution of saline. Techniques for formulation and administration of drugs may be found in “Remington's Pharmaceutical Sciences,” Mack Publishing Co., Easton, Pa., latest edition.

One may administer the pharmaceutical composition in a systemic manner (as detailed hereinabove). Alternatively, one may administer the pharmaceutical composition locally, for example, via injection of the pharmaceutical composition directly into a cardiac tissue region of a subject.

Pharmaceutical compositions of the present disclosure can be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present disclosure thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

Depending on the particular cardiac disease or condition, the subject may be administered with additional chemical drugs (e.g., immunomodulatory, chemotherapy etc.) or cells.

In one embodiment, the plurality of solid dECM particles can be administered as a co-therapy to further promote ventricular unloading. In such instances, a plurality of solid dECM particles can be administered prior to, simultaneous with, or following treatment with a ventricular unloading modality. In one example, the ventricular unloading modality can include a mechanical unloading device, such as left ventricular assist device (LVAD). In another example, the ventricular unloading modality can include a chemical or drug, such as a systemic vasodilator (e.g., nitroprusside, phentolamine, nitroglycerin). Advantageously, the combined administration of solid dECM particles and a ventricular unloading modality can reduce oxygen demand and limit infarct size to thereby treat (e.g., prevent or mitigate) ventricular remodeling.

Doses for subjects (e.g., humans) can be determined without undue experimentation by the skilled artisan, from this disclosure and the knowledge in the art. A dose of solid dECM particles appropriate to be used in accordance with various embodiments of the present disclosure will depend on numerous factors. The parameters that will determine optimal doses to be administered for primary and adjunctive therapy generally will include some or all of the following: the cardiac condition or disease being treated and its stage; the species of the subject, their health, gender, age, weight, and metabolic rate; the subject's immunocompetence, if any; other therapies being administered; and expected potential complications from the subject's history or genotype. Additional parameters can include co-administration with other factors (such as a ventricular unloading modality). The optimal dose in a given situation also will take into consideration the way in which the solid dECM particles are formulated and the way they are administered.

In various embodiments, solid dECM particles can be administered in an initial dose, and thereafter maintained by further administration. Solid dECM particles may be administered by one method initially, and thereafter administered by the same method or one or more different methods. The levels can be maintained by the ongoing administration of the solid dECM particles. Various embodiments administer the solid dECM particles either initially or to maintain their level in the subject, or both, by direct or intravenous injection. In a variety of embodiments, other forms of administration are used, dependent upon the subject's condition and other factors, discussed elsewhere herein.

Solid dECM particles can be administered in many frequencies over a wide range of times. Generally, lengths of treatment will be proportional to the length of the disease process, the effectiveness of the therapies being applied, and the condition and response of the subject being treated.

Another aspect of the present disclosure can include a method for preserving a donor heart intended for heart transplant. In one embodiment, a plurality of solid dECM particles of the present disclosure can be administered onto and/or into a donor heart prior to, concurrent with, and/or following surgical implantation of the donor heart into a recipient subject. Advantageously, use of the solid dECM particles of the present disclosure can mitigate ischemia and promote angiogenesis in the donor heart post-transplantation.

### Exemplary Aspects

In view of the described compositions, devices, and methods and variations thereof, herein below are certain more particularly described aspects of the present disclosure. These particularly recited aspects should not, however, be interpreted to have any limiting effect on any different claims containing different or more general teachings described herein, or that the “particular” aspects are somehow limited in some way other than the inherent meanings of the language literally used therein.

Aspect 1: A solid particle comprising decellularized cardiac tissue extracellular matrix.

Aspect 2: The solid particle of Aspect 1, being a microparticle.

Aspect 3: The solid particle of any one of Aspects 1-2, having a diameter of about 1 μm to about 100 μm.

Aspect 4: The solid particle of any one of Aspects 1-3, having a diameter of about 20 μm.

Aspect 5: The solid particle of any one of Aspects 1-4, being spherical.

Aspect 6: The solid particle of any one of Aspects 1-5, wherein the cardiac tissue is human or porcine.

Aspect 7: The solid particle of any one of Aspects 1-6, wherein 100% of the particle is composed of decellularized cardiac tissue extracellular matrix.

Aspect 8: The solid particle of any one of Aspects 1-7, not being a liquid, hydrogel, or sol-gel ex vivo.

Aspect 9: The solid particle of any one of Aspects 1-8, having an elastic modulus of about 1.5 kPa to about 6.5 kPa.

Aspect 10: The solid particle of any one of Aspects 1-9, having an elastic modulus greater than about 2.5 kPa, about 3 kPa, about 3.5 kPa, about 4 kPa, about 4.5 kPa, about 5 kPa, about 5.5 kPa, about 6 kPa, about 6.5 kPa, or greater than about 2.5 kPa but less than about 6.5 kPa.

Aspect 11: The solid particle of any one of Aspects 1-10, having an elastic modulus of about 4.5 kPa.

Aspect 12: The solid particle of any one of Aspects 1-11, when exposed to or immersed in an aqueous environment, swells up to about 50-90% as compared to a hydrogel or a hydrogel precursor in a non-aqueous environment.

Aspect 13: The solid particle of any one of Aspects 1-12, when exposed to or immersed in an aqueous environment, swells up to about 70% as compared to a hydrogel or a hydrogel precursor in a non-aqueous environment.

Aspect 14: The solid particle of any one of Aspects 1-13, being resistant to complete enzymatic degradation about 1.5 times, about 2 times, about 2.5 times, about 3 times, about 3.5 times, about 4 times, about 4.5 times, about 5 times, about 5.5 times, about 6 times, about 6.5 times, about 7 times, about 7.5 times, about 8 times, about 8.5 times, about 9 times, about 9.5 times, about 10 times, about 10.5 times, or about 11 times greater than a time for complete enzymatic degradation of a hydrogel or hydrogel precursor.

Aspect 15: The solid particle of any one of Aspects 1-14, being resistant to complete enzymatic degradation for about 24 hours.

Aspect 16: The solid particle of any one of Aspects 1-15, wherein the solid particle, in a physiological environment, releases one or more biomolecules at a controlled release rate over a period of time.

Aspect 17: The solid particle of Aspect 16, wherein the period of time is about 24 hours, about 24-28 hours, about 28-32 hours, about 32-36 hours, about 36-40 hours, about 40-44 hours, or about 44-48 hours.

Aspect 18: The solid particle of Aspect 17, wherein the period of time is about 24 hours.

Aspect 19: The solid particle of any one of Aspects 1-18, wherein the solid particle, in a physiological environment, releases one or more biomolecules at a controlled release rate of about 0.5 ppm/hour to about 4 ppm/hour, or about 0.5-1 ppm/hour, or about 1-1.5 ppm/hour, or about 1.5-2 ppm/hour (e.g., 2 ppm/hour), or about 2-2.5 ppm/hour, or about 2.5-3 ppm/hour, or about 3-3.5 ppm/hour, or about 3.5-4 ppm/hour, or about 4-4.5 ppm/hour, or about 4.5-5 ppm/hour.

Aspect 20: The solid particle of any one of Aspects 1-19, wherein the solid particle, in a physiological environment, releases one or more biomolecules at a controlled release rate of about 2 ppm/hour.

Aspect 21: The solid particle of any one of Aspects 1-20, wherein the solid particle, in a physiological environment, releases one or more biomolecules at a controlled release rate of about 35 ppm over 24 hours to about 45 ppm over 24 hours, or about 35-36 ppm over 24 hours, or about 36-37 ppm over 24 hours, or about 37-38 ppm over 24 hours (e.g., about 38 ppm over 24 hours), or about 38-39 ppm over 24 hours, or about 39-40 ppm over 24 hours, or about 40-41 ppm over 24 hours, or about 41-42 ppm over 24 hours, or about 42-43 ppm over 24 hours, or about 43-44 ppm over 24 hours, or about 44-45 ppm over 24 hours.

Aspect 22: The solid particle of any one of Aspects 1-21, wherein the solid particle, in a physiological environment, releases one or more biomolecules at a controlled release rate of about 38 ppm over 24 hours.

Aspect 23: The solid particle of any one of Aspects 1-22, wherein no more than about 20-50% of total protein is released from the solid particle within about 20-50 hour(s) following contact of the solid particle with a physiological environment.

Aspect 24: The solid particle of any one of Aspects 1-23, wherein no more than about 20%, no more than about 25%, no more than about 30%, no more than about 35%, no more than about 40% (e.g., no more than about 37%), no more than about 45%, or no more than about 50% of total protein is released from the solid particle within about 20 hours, about 25 hours, about 30 hours, about 35 hours, about 40 hours, about 45 hours, or about 50 hours (e.g., about 48 hours) following contact of the solid particle with a physiological environment.

Aspect 25: The solid particle of any one of Aspects 1-24, wherein no more than about 37% of total protein is released from the solid particle within about 48 hours following contact of the solid particle with a physiological environment.

Aspect 26: The solid particle of any one of Aspects 1-25, further comprising at least one biomolecule that is chemically conjugated thereto.

Aspect 27: The solid particle of Aspect 26, wherein the at least one biomolecule is a non-native biomolecule.

Aspect 28: The solid particle of any one of Aspects 26-27, wherein the biomolecule is a macromolecule or a small molecule.

Aspect 29: The solid particle of any one of Aspects 26-28, wherein the macromolecule is a macromolecule-loaded polymer.

Aspect 30: The solid particle of any one of Aspects 26-29, wherein the macromolecule or small molecule is one of a drug or a drug-loaded nanoparticle.

Aspect 31: The solid particle of any one of Aspects 26-30, wherein at least one surface of the solid particle is functionalized with one or more chemical and/or biological moieties.

Aspect 32: The solid particle of any one of Aspects 1-31, being formed by electrospraying.

Aspect 33: The solid particle of any one of Aspects 1-31, being formed by use of a microfluidic chip.

Aspect 34: A composition comprising the solid particle of any one of Aspects 1-31 and a second component (e.g., an additive, vehicle or carrier).

Aspect 35: A pharmaceutical composition comprising the solid particle of any one of Aspects 1-31 and a pharmaceutically acceptable carrier.

Aspect 36: A patch comprising a biodegradable bioscaffold seeded with a plurality of the solid particles of any one of Aspects 1-31.

Aspect 37: A solid particle consisting of decellularized cardiac tissue extracellular matrix.

Aspect 38: A solid particle consisting of decellularized cardiac tissue extracellular matrix and at least one biomolecule chemically conjugated to the extracellular matrix.

Aspect 39: A method of treating a cardiac disease or cardiac condition in a subject, comprising administering to the subject a therapeutically effective amount of the solid particles of any one of Aspects 1-31, thereby treating the cardiac disease or cardiac condition.

Aspect 40: The method of Aspect 39, wherein the subject has experienced a myocardial infarction prior to administration of the plurality of solid particles.

Aspect 41: The method of any one of Aspects 39-40, wherein, following administration of the plurality of solid particles, one or more of the following effects occurs in the subject: (1) preservation of cardiac function; (2) reduction in fibrosis and fibroblast activation; (3) stimulation of cardiomyocyte cell cycle activity; (4) angiogenesis in cardiac tissue; and (5) protection against ventricular wall remodeling.

Aspect 42: The method of any one of Aspects 39-41, wherein the plurality of solid particles is administered prior to, simultaneous with, or following treatment with a ventricular unloading modality.

The following Example is for the purpose of illustration only and is not intended to limit the scope of the claims, which are appended hereto.

### Example

This Example describes an experiment in which solid dECM microparticles were generated by electrospray and heat-induced gelation. The generated dECM microparticles were characterized for protein release kinetics, swelling capacity, stiffness, digestion resistance, and stability. Cardiac function, cardiomyocyte cell cycle activity, fibrosis, fibroblast activation, and angiogenesis were investigated in post-MI hearts of mice. As detailed below, it was surprisingly discovered that the generated solid dECM microparticles exhibited extended stability in cardiac tissue and controlled protein release to improve the heart post-injury response.

### Methods

dECM Isolation and Solubilization

dECM harvest was based upon previous publications. Porcine tissue was acquired for secondary use from animals on protocols approved by the Case Western Reserve University (CWRU) Institutional Animal Care and Use Committee (IACUC). Animals were first anesthetized by an intramuscular injection of Telazol and then euthanized by an overdose of Fatal-Plus, pentobarbital sodium (>100 mg kg⁻¹). This method is consistent with the recommendations of the 2000 Panel on Euthanasia of the American Veterinary Medical Association. Porcine ventricles were cut to approximately 2 cm³ dices and immersed in 1% sodium dodecyl sulfate solution (Sigma-Aldrich, St. Louis, MO, USA) to decellularize for 48 h until tissue appeared white in color. The samples were washed and soaked in a 1% solution of Triton X-100 (Sigma-Aldrich) for 4 h. The samples were then washed three times with deionized water for 12-24 h. Samples were lyophilized (SP Scientific, Gardiner, NY, USA) and kept at −80° C. for long-term storage. To generate dECM solution, lyophilized dECM was pulverized in liquid nitrogen and digested in 1 mg mL⁻¹ pepsin (Sigma-Aldrich) in 0.01 M HCl buffer for 10 h at room temperature. Next, dECM was added at 10 mg mL⁻¹ digestion buffer. Once completed, the pH was adjusted to 7 by adding 1 N of NaOH, and then the sample was diluted in 1:10 by volume in 10× phosphate buffered saline (PBS; pH˜7.4). Penicillin-streptomycin (P/S) (10,000 U mL⁻¹, Cytiva, Marlborough, MA, USA) was added to reach a final concentration of 100 U mL⁻¹. The sample was then stored at −20° C. The dECM solution is a hydrogel precursor that aggregates into a 3D hydrogel at physiological temperature such as when administered in vivo.

Solid dECM Microparticle Fabrication

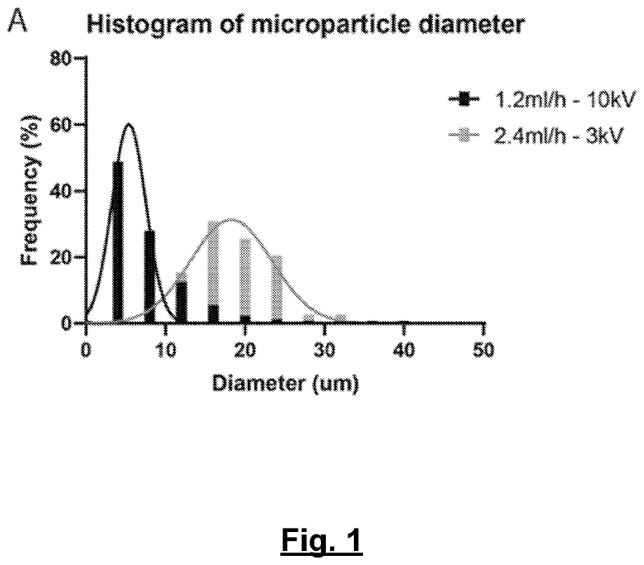

The solid dECM microparticles were fabricated using an electrospray apparatus (Spraybase, Cambridge, MA, USA) (FIG. 1A). Solubilized dECM solution was loaded into a 5 mL syringe (BD, Franklin Lakes, NJ, USA) and a syringe pump (New Era Pump Systems, Inc. Farmingdale, NY, USA) was set to 1.2 mL per hour flow rate. The syringe was connected to tubing which terminated in a 23-G needle. A high-power electrical source was connected to a needle and then separately the ground collection vessel via copper tape. A stir bar was placed inside the collection vessel and set to 100 RPM. The collection vessel is then filled with 7 mL olive oil with 5% surfactant (24% Tween 85 and 76% Span 85; Sigma-Aldrich). Needle distance is aligned to the beaker height. Syringe pump was then started and the voltage was set to between 8 kV to 17 kV depending on the intended sample. Once spraying is completed, the sample is transferred to another beaker with constant stirring at 600 RPM on a hot plate at 50° C. for 2 h in a desiccator. After heat-induced gelation, microparticles were washed with 3 mL acetone (Sigma-Aldrich) three times, spun at 1,700 g with each supernatant discarded, and then in 3 mL ethanol (Decon labs, Inc., King of Prussia, PA, USA) three times, with each spun at 1,700 g discarding the supernatant each time. The samples were suspended in ethanol and stored at −20° C. The final dECM microparticles have a concentration ˜10 mg mL⁻¹.

### Protein Release Assay

For release kinetics, the dECM microparticles (˜1 mg) and gelled dECM hydrogel precursor 100 μL (˜1 mg) were incubated in 600 μL of 1×PBS (37° C.). In the first 24 h, 60 μL buffer was sampled at 4 min, 15 min, 30 min, 1 h, 2 h, 4 h, 8 h, and 24 h. Fresh 1×PBS was added after sampling. After 24 h, the entire buffer was sampled at days 2, 4, 6, 8, 10, 12, and 14. Fresh 1×PBS was used to replace the old buffer after sampling. Samples were kept at −20° C. BCA assay (ThermoFisher, Waltham, MA, USA) was used to measure the protein concentrations.

### Swelling Analysis

The dECM microparticles and liquid-derived dECM hydrogels were imaged using a Zeiss fluorescence microscope (White Plains, NY, USA) in phase contrast mode. To evaluate swelling, the microparticles and hydrogels in ethanol were placed on charged glass slides (Fisher Scientific, Pittsburgh, PA, USA) and allowed to partially dry and adhere to the glass slide. A coverslip was gently placed on top, and ethanol was allowed to perfuse the area to re-solvent microparticles. After 10 min, the solution was exchanged with distilled (DI) water showing the change in size from the ethanol solvent to water after 15 min.

### Water Uptake

The dECM microparticles and liquid-derived dECM hydrogels were evaluated for water uptake following a published protocol. To quantify changes in mass due to water uptake, the mass of microcentrifuge tubes was individually measured for baseline weight. The dECM microparticles and hydrogels were then added to the microcentrifuge tubes and weighed to get an initial “wet weight.” The samples were then lyophilized and weighed for the “dry weight.” Then water was added in 40 μL increments to rehydrate the samples and make sure that no excess water was added. If there was excess water, it was removed via pipette before weighing again. These masses were subtracted from each other to get changes in mass and percentage changes.

### Scanning Electron Microscopy

Scanning electron microscope (SEM, FEI Helios FIB, ThermoFisher) was used for imaging and surface analysis of dECM microparticles and liquid-derived hydrogels. Samples were suspended and fixed in 2.5% glutaraldehyde (Sigma-Aldrich) and 2% paraformaldehyde (PFA) (Sigma-Aldrich) in 0.1 M cacodylic acid (Sigma-Aldrich) for 1 h at room temperature. dECM microparticles and hydrogels were washed with 0.1 M cacodylic acid twice and were then serially dehydrated in ethanol (20, 50, 70, 90, 100% ethanol). After dehydration, the samples were pipetted onto glass coverslips and were left to air dry at room temperature overnight. The dried samples were then sputter-coated (Denton Vacuum, LLC., Moorestown, NJ, USA) with platinum before SEM imaging.

### Collagenase Digestion

One hundred microliters of dECM hydrogel and microparticles suspended in PBS were added to 1.5 mL centrifuge tubes. The mass of the sample in each tube is 1 mg. Seven-hundred microliter of fresh collagenase (Sigma-Aldrich) digestion buffer (0.1 mg mL⁻¹ collagenase and 0.36 mM CaCl₂ in 1×PBS) was added to each tube. Samples were incubated in a 37° C. incubator. Seventy-two microliter of digestion buffer was sampled at time 0, 15, 30, 60, 120, 240, 480, and 1440 minutes, and the same volume of fresh collagenase digestion buffer was added. Protein concentrations were measured using BCA assay.

### Microparticle Retention and Stability Assay

After washing in 1×PBS three times, dECM microparticles and dECM hydrogel precursor were labeled with 100 μg mL⁻¹ wheat germ agglutinin-Alexa fluor 488 (ThermoFisher) for 2 h at 4° C. with constant agitation. Two microliters of dECM hydrogel precursor and equivalent mass of dECM microparticles were injected through a 10 μL Hamilton syringe into a biopsy core of ˜3 mm×3 mm×2 mm porcine heart cube before plating into a 48-well tissue culture dish. Samples were incubated at 37° C. for 2 h to induce dECM hydrogel precursor gelation without potential washout from buffer. Samples were then incubated in 500 μL DMEM media (ThermoFisher) for 14 days. Media was changed every 3 days. Samples were washed, cryosectioned on days 1, 3, 7, and 14, and imaged immediately. To measure the area of fluorescence-labeled dECM, images were quantified using ImageJ (NIH, USA). Histogram of pixel intensity was used to determine the threshold of non-tissue area, tissue-background, and Alexa Fluor 488-labeled signal of dECM. To measure the relative fluorescence intensity, images were measured with the defined thresholds for the averaged intensity of tissue and dECM regions of interest, and then label fluorescence intensities were normalized to the tissue.

### Atomic Force Microscopy (AFM)

A MFP-3D-Bio AFM (Asylum Research, Oxford Instruments, Santa Barbara, CA, USA) mounted on an inverted fluorescence microscope (Nikon Eclipse Ti) was used to indent the dECM hydrogels or microparticles and obtain their Young's modulus. The dECM microparticles suspended in ethanol were coated on clean glass slides and ethanol was allowed to evaporate (˜2 h) prior to testing. Similarly, dECM hydrogels were placed in sterile AFM Petri dishes (Corning; 35 mm×10 mm) and covered in PBS (37° C.). Tip-less AFM cantilevers (model Arrow TL 1, Nanoworid, nominal spring constant ˜0.02 N m⁻¹) were glued with a 2±0.05 μm diameter polystyrene bead (Sigma) using epoxy. The spring constant was determined from a force-distance curve and using thermal calibration method in a clean culture dish containing PBS (for gel analysis) or on a clean glass slide (for microparticle analysis). Multiple force curves were obtained at random locations on the hydrogels immersed in PBS at a rate of ˜5.1 μm s⁻¹ and a trigger force of ˜4 nN. The resulting force versus indentation curves were analyzed by Hertz model fit for spherical indenters using Igor Pro 6.37 software. The Young's modulus was determined from these force curves as F=4Eγ√Rδ³/3(1−μ²), where F is indentation force, EΥ is Young's modulus, μ is Poisson's ratio (˜0.4), R is tip radius (1 μm) and δ is the indentation depth (˜500 nm). Data were represented as mean±standard deviation from at least n=6 independent gels/well or multiple particles per glass slide, with at least three independent repeats of each experiment.

### Mice MI Model and Echocardiography

All animal procedures were reviewed and approved by IACUC at CWRU according to the guidelines and regulations described in the Guide for the Care and Use of Laboratory Animals (National Academies Press, 2011).

Myocardial infarction was performed on non-regenerative day 5 mice as previously published (X. Wang et al., Acta biomaterialia, vol. 113, pp. 380-392, September 2020; X. Wang et al., J Mol and Cell Cardiology, vol. 0, no. 0, June 2021). Pregnant CD1 (IGS) mice were purchased from Charles River (Wilmington, MA, USA). Day 5 mice were anesthetized by hypothermia and kept on ice during the procedure. Analgesia was not available because of the age of the mice. Anesthesia was monitored by toe-pinch every 15 min if the procedures are above 15 min. An incision was made on the fourth intercostal space, pericardium was removed to better visualize coronary arteries. A 10-0 Nylon suture (Arosurical instruments co., Newport Beach, CA, USA) was used to permanently ligate left coronary artery. Two microliters of dECM hydrogel precursor and dECM microparticles were injected through a 10 μL Hamilton syringe into myocardium. Two injections were made above and below the ligation site. Wound was closed by suture and skin glue (Henry Schein, Melville, NY, USA). Recovered mice were returned to the dame. Three weeks post-MI, mice were anesthetized by 5% isoflurane (Patterson veterinary, Greeley, CO, USA) vapor in air before echocardiography and maintained anesthetized using 1% to 1.5% isoflurane as previously published (M L Lindsey et al., Am J Phys-Heart Circl Phys, vol. 314, no. 4, pp. H733-H752, April 2018). Body temperature was maintained at 37° C. and heart rate was kept at 400 to 500 beats per minute. M-mode and B-mode were recorded along the long axis using a Vevo 3100 (VisualSonics) equipped with a MX550D transducer. Cardiac function was analyzed using Vevo lab (VisualSonics). BrdU was injected into mice at 0.1 mg g-body weight for 3 days before euthanization. Animals were euthanized without anesthesia by introducing 100% C02 to their home cage for 10 min. The C02 flow rate was about 20% of chamber volume per minute. Death was confirmed by heart removal.

### Histology and Immunostaining

For Masson's Trichrome staining, heart sections were deparaffinized and stained as per protocols provided by the vendor (Electron Microscopy Sciences, Hatfield, PA, USA). For immunostaining, after deparaffinization and antigen retrieval, sections were blocked in 10% serum buffer for 1 h at room temperature and incubated in respective primary antibody staining buffers overnight at 4° C. Rabbit anti-platelet derived growth factor receptor alpha (Pdgfr-α) (Abclonal, Woburn, MA, USA), chicken anti-Vimentin (Abcam, Waltham, MA, USA), rabbit anti-phospho-histone H3 (PHH3) (Abcam), mouse anti-alpha smooth muscle actin (α-SMA) (ThermoFisher), mouse anti-cardiac troponin T (TnT) (Developmental Studies Hybridoma Bank, Iowa City, IA, USA), rabbit anti-Ki67 (ThermoFisher), rabbit anti-BrdU (ThermoFisher), and rabbit anti-CD31 (Abcam) antibodies were used. Fluorophore-conjugated goat secondary antibodies (ThermoFisher) were used for detecting primary antibodies. Nuclei were stained by DAPI (Sigma-Aldrich). Sampled were mounted in anti-fade mounting media (Vectoriab, Burlingame, CA, USA) and imaged within 2 days.

For fluorescent immunostaining, MI groups' cardiomyocytes in the border area (within 250 μm from the edge of infarct area) were imaged. Fibroblasts in infarct and border areas (within 100 μm of collagen-rich infarct area and in the infarct area) and vessels in infarct and border areas (within 400 μm of collagen-rich infarct area and in infarct area) were imaged. Cardiomyocytes, fibroblasts, and vessels in sham group were randomly imaged in left ventricle wall. For each section, 5 images were randomly taken in the area mentioned above. Cell numbers were counted in imageJ by thresholding using Auto Threshold (Moments, Otsu, or Percentile methods). The segmented color channel of DAPI and the channel of specific cell markers were overlayed to highlight specific cells of interest. Cell number was counted using Analyze Particle function. BrdU, Ki67, α-SMA positive cells, and CD31—α-SMA double positive structures were counted manually. For Masson's Trichrome staining, the whole heart section was imaged and measured using imageJ Auto Thresholding. Fibrotic area was measured by Auto Thresholding and manual choosing the infarct region.

### Microparticle Macroparticle Incorporation and Conjugation

As a proof-of-concept for drug loading, dECM microparticles were fabricated after doping with latex beads and with dextran. Carboxylate-modified latex beads (ThermoFisher; 200 nm diameter; 2% w v⁻¹ suspension) were mixed at 1:10 ratio with the dECM hydrogel precursor before electrospray. Alternatively, fluorescein isothiocyanate-dextran 10 kDa (Sigma-Aldrich) were enriched in the dECM hydrogel precursor to reach final concentrations of 0.1 mg mL⁻¹ or 1 mg mL⁻¹. The doped dECM mixture was then processed by the microparticle fabrication protocol by electrospray and heat-induced gelation. Doped dECM microparticles were washed in acetone, ethanol, and 1×PBS three times each, before dextran release experiments. Microsphere incorporation and dextran loading were evaluated using fluorescence microscopy.

To determine dextran release from microparticles, dextran loaded dECM microparticles (1 mg) were incubated in 400 μL PBS at 37° C. for 7 days with daily exchange of buffer. Dextran concentrations were examined by Synergy H1 spectrometry (BioTek Instruments, Winooski, VT, USA) using 490 nm excitation wavelength and 520 nm emission wavelength.

Surface functionalization was conducted for proof-of-concept of tissue targeting and surface modular motifs. NHS-AF488 (Lumiprobe, Hunt Valley, MD, USA) stock solution was added to dECM microparticles suspended in 0.1 M sodium bicarbonate buffer. Fifty micrograms of NHS-AF488 were used per milligram of dECM microparticles. dECM microparticles were incubated with NHS-AF488 at 4° C. overnight, followed by washing in PBS five times. Surface functionalized dECM microparticles were imaged using a fluorescence microscope.

dECM Digestion and Liquid Chromatography and Mass Spectrometry

Lyophilized and pulverized dECM samples were digested by trypsin protease, MS grade (ThermoFisher) in 1×PBS buffer. One milligram of dry dECM was digested by 50 μg trypsin in 1 ml PBS. Six hundred nanograms of each sample were analyzed using Dionex Ultimate 3000 capillary LC system (Dionex, Sunnyvale, CA, US) and LTQ-Orbitrap XL (Waltham, MA, US). Raw data were first processed using Proteomarker software v1 (Inforchromics, Toronto, Canada), and then searched via Mascot v2.2 (Matrix Science, London, UK) against Proteomes—Sus scrofa (UniProt, Switzerland). An expectation value of p≤0.05 and an estimated false discovery rate of 1% were used. Proteins had more than 2 identified peptides were used for further analysis. Protein locations were annotated manually. The numbers of identified peptides were used to determine the abundance of extracellular matrix proteins.

### Microparticles Sonication and Bead-Homogenization

For sonication, liquid dECM droplets suspended in oil after electrospray were transferred to conical tubes and sonicated in a 0.8 L bath sonicator (Btihceuot) for 30 min using 42 kHz sonication frequency and 35 W power. Ice-cold water was used to keep the temperature at 0° C.

For bead-homogenization, solid dECM microparticles were suspended in ethanol with polystyrene beads and metal beads for centrifugal agitation. Then washed in acetone three times (5 min each), and three more times (5 min each) in ethanol for cold storage, or switched to aqueous buffer for cell treatment.

### Fourier-Transform Infrared Spectroscopy

dECM microparticles were dried on a coverslip and transferred to a Cary 630 FTIR facility (Agilent, Santa Clara, CA, USA) for reading. Spectrum range was set to 4000 to 600 per centimeter with a resolution of 4/cm. Background calibration was conducted before reading a new sample. Averaged data of 512 scans was used for analysis. Baseline correction and peak normalization were used for processing FTIR data.

### Cardiomyocyte Cell Cycle Activity in Heart Explants

All animal procedures were reviewed and approved by IACUC at CWRU according to the guidelines and regulations described in the Guide for the Care and Use of Laboratory Animals (National Academies Press, 2011).

Hearts of day 1 CD1(IGS) neonatal mice were harvested and washed in 1×PBS. Animals were euthanized by decapitation using surgical scissors without anesthesia according to Animal Research Advisory Committee Guidelines (NIH Office of Intramural Research, 2020). Ventricles were cut to ˜1 mm³ pieces using surgical scissors. Two microliters of dECM microparticles or dECM hydrogel precursor were injected through 10 μL Hamilton syringes into mouse explants before plating explants on 1.5 mg mL⁻¹ collagen (Corning) hydrogels in 48-well plate dish. After incubating at 37° C. for 2 h, explants were cultured in M199 supplemented with 1% fetal bovine serum (FBS; ThermoFisher), 1× insulin-transferrin-selenium (ITS; Corning), 2 mM L-glutamine (ThermoFisher), and 1× penicillin-streptomycin (P/S; ThermoFisher; explant culture media] for 6 days. Media was changed every 3 days. Cell cycle activity was labeled by 3-day BrdU (Sigma-Aldrich) labeling. Explants were washed in 1×PBS twice and fixed in 4% PFA solution overnight before cryosection.

### Statistical Analysis

Statistical analysis was conducted using GraphPad Prism 7. Data were represented as bar graph or dot plot with mean±standard deviation (SD). Number of experimental repeats is shown in figure legends. A student t-test was performed to compare experiments with two groups. One-way ANOVA and Tukey's test were used for analyzing experiments with three or more groups. The confidence interval was set to 0.95. In vivo experiments were conducted on four individual animals for each treatment, while in vitro experiments were repeated independently three times unless stated otherwise. For cardiomyocytes, 300 to 550 cardiomyocytes per animal, or 1,200 to 2,200 cardiomyocytes per treatment group were analyzed. For fibroblasts, 50 to 100 cells per animal in sham group and 120 to 320 cells in MI groups, or 200 to 400 fibroblasts in sham group and 480 to 1280 fibroblasts in MI groups were analyzed.

### Results

Solid dECM Microparticle Generation

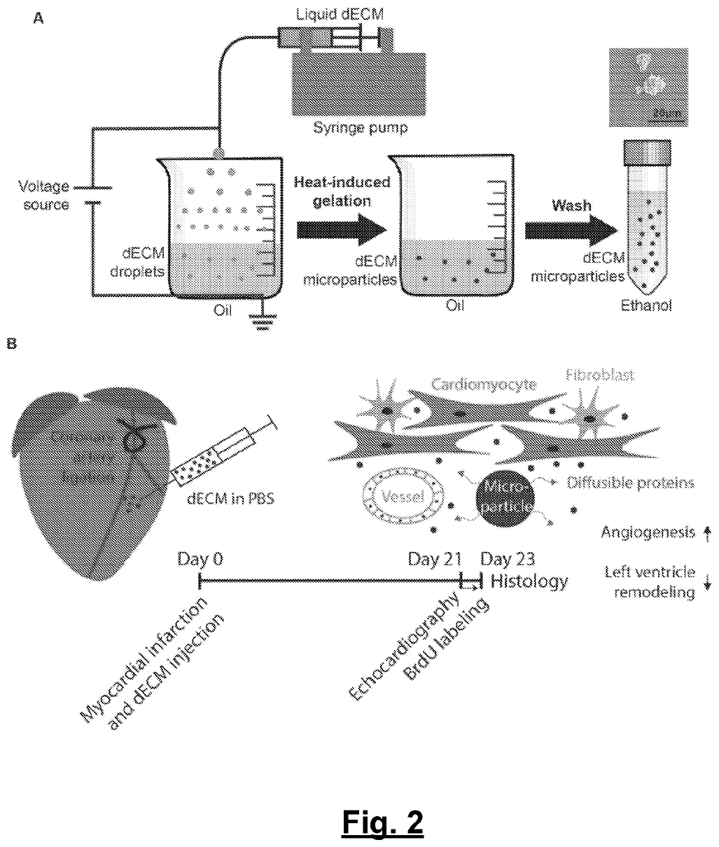

The dECM microparticles were generated by electrospray of solubilized dECM (FIG. 2A). The particle size can be controlled by tuning electrospray voltage and syringe pump flow rate (FIG. 1A). The microparticles used in this study were approximate 20 μm in diameter.

The components of dECM were analyzed by liquid chromatography-mass spectrometry of trypsin digested dECM (Table 1).

TABLE 1
Top 20 matrix proteins in dECM

Collagen type VI sub units
Fibronectin
Heparan sulfate proteoglycan 2
Laminin subunits
Fibrinogen subunits
Periostin
Transglutaminase 2
Tenascin X
Inter-alpha-trypsin inhibitor subunits
Collagen type XIV subunits
Elastin microfibril interfacer 1
Agrin
Alpha(B)-crystallin
Collagen type I subunits
Elastin
C3a anaphylatoxin
Fibrillin-1
Apolipoprotein A1
Plasminogen
Collagen type XII alpha 1 chain

The dECM contains extracellular matrix structural proteins like collagen I, fibronectin, laminin, and fibrillin. Growth factors directing proliferation and differentiation such as agrin and periostin, and angiogenesis factors such as heparan sulfate proteoglycan 2 and alpha(B)-crystallin were also identified in dECM. Thus, the proteome of dECM microparticles has sensitivity to proteases in the infarct area and can influence cardiac cell phenotypes by macromolecules released by passive diffusion and degradation (FIG. 2B).

dECM Microparticles Stimulate Angiogenesis by 3 Weeks in Post-MI Hearts

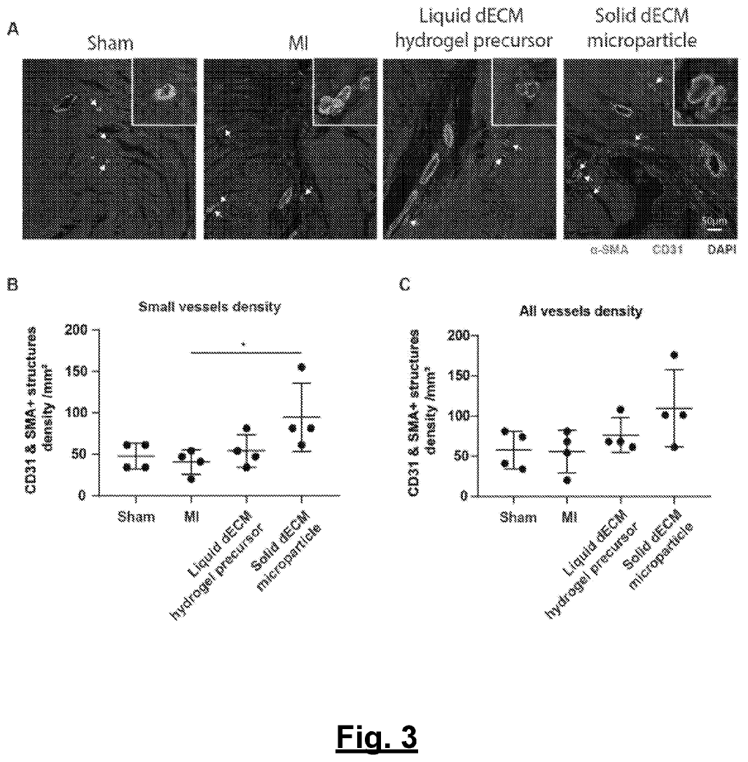

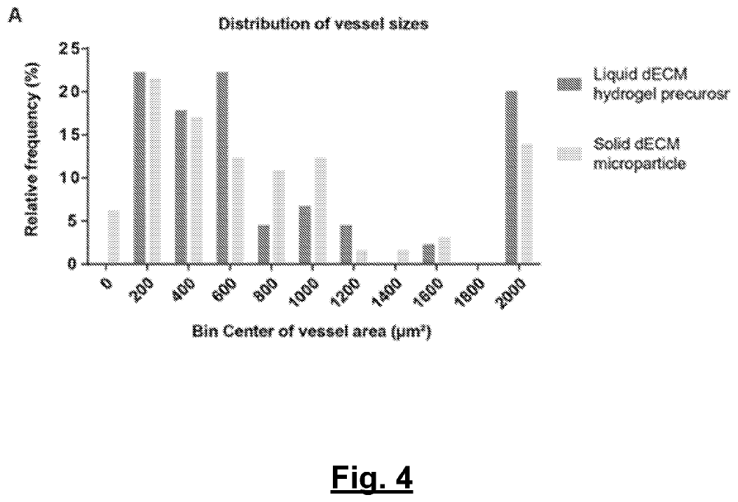

The dECM treatments were evaluated as a therapy for the post-ischemic heart. After ligating left coronary artery in day 5 mice hearts, vessels densities at week 3 post-MI were examined by fluorescent immunostaining (FIG. 3A). Blood vessels were identified as α-SMA (smooth muscle) and CD31 (endothelial) double-positive structures in this study. To better investigate the newly-formed vessels caused by angiogenic activity, blood vessel structures below 1,000 μm² were analyzed. The threshold reflects an inflection in the histogram data of vessel areas (FIG. 4A). The dECM microparticles significantly increased the density of small CD31 and α-SMA positive structures compared to MI-control (FIG. 3B) unlike the dECM hydrogel precursor. dECM microparticles treatment also indicated a trend of increasing the density of all vessels (FIG. 3C). The results indicate that dECM microparticles can distinctly increase small blood vessel growth in post-MI hearts.

dECM Microparticles Preserve Cardiac Output 3 Weeks Post-MI

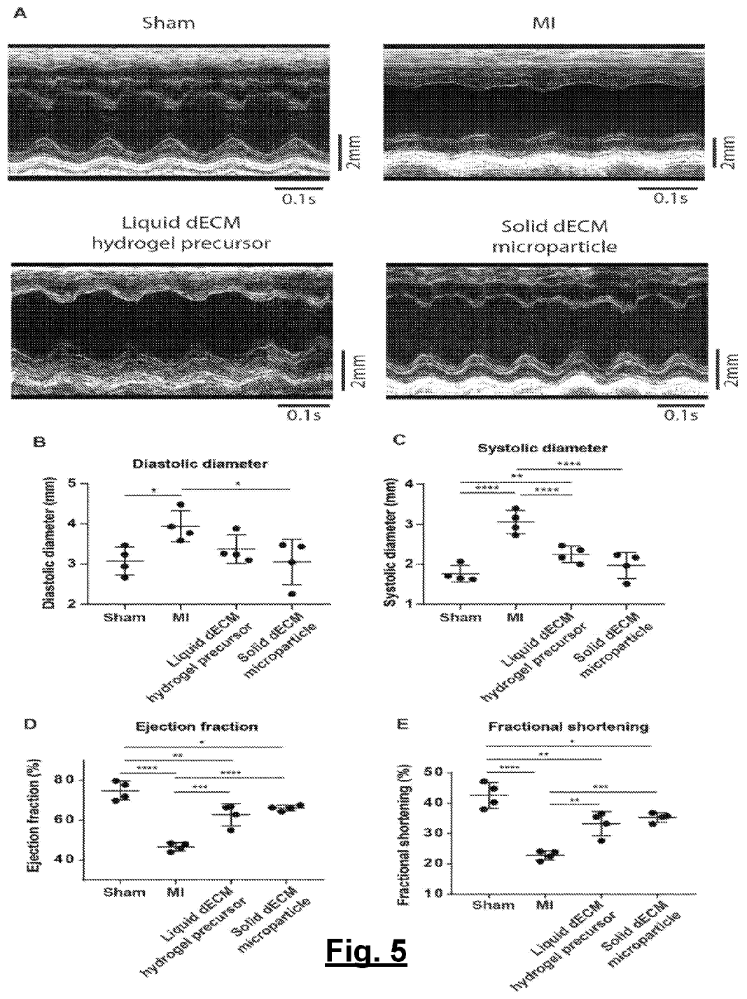

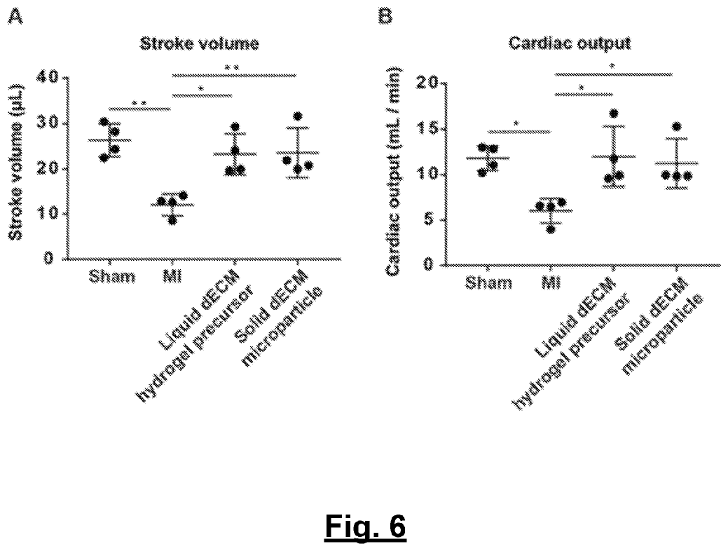

Cardiac function was measured by echocardiography at week 3 post-MI (FIG. 5A). dECM microparticles lowered left ventricle end diastolic diameter compared to MI-control unlike the dECM hydrogel precursor (FIG. 5B). Both dECM microparticles and dECM hydrogel precursor lowered end systolic diameter compared to MI-control, but dECM microparticles treated hearts was not significantly different from sham levels (FIG. 5C). The ejection fraction (FIG. 5D) and fractional shortening (FIG. 5E) were higher with both dECM treatments relative to MI-control. The stroke volume (FIG. 6A) and cardiac output (FIG. 6B) were also higher in dECM microparticles and dECM hydrogel precursor treated hearts in comparison to the MI-control. Together, the results demonstrate that dECM microparticles and dECM hydrogel precursor exhibit comparable protective effects on cardiac function in post-MI hearts, while dECM microparticles show a higher protection against left ventricular remodeling.

dECM Microparticles Reduce Fibrosis and Fibroblasts Activation 3 Weeks Post-MI

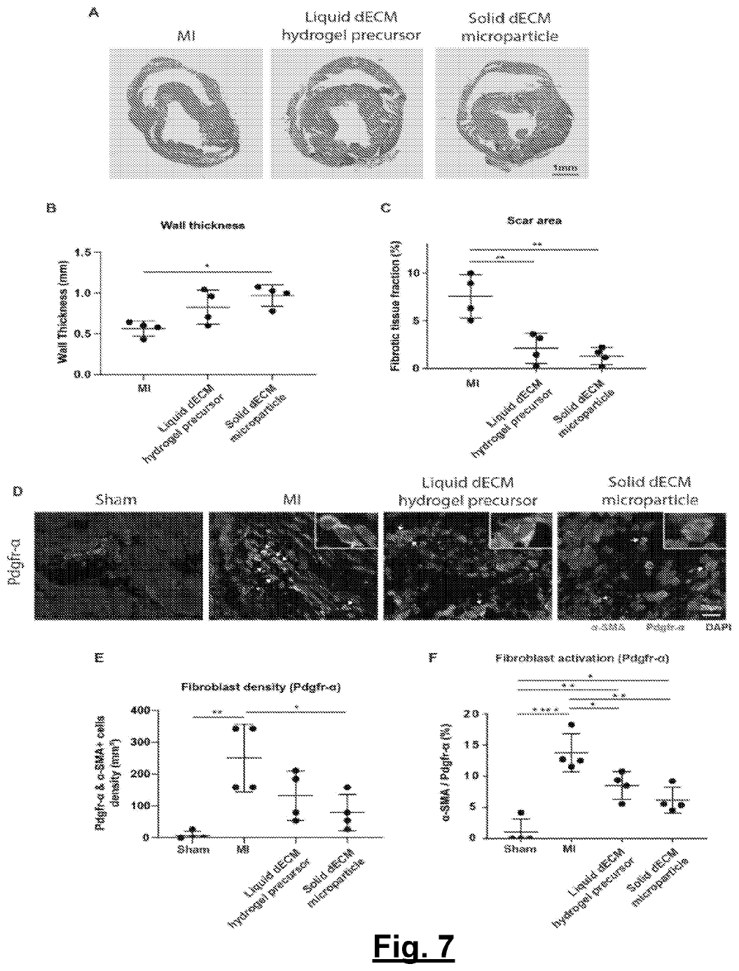

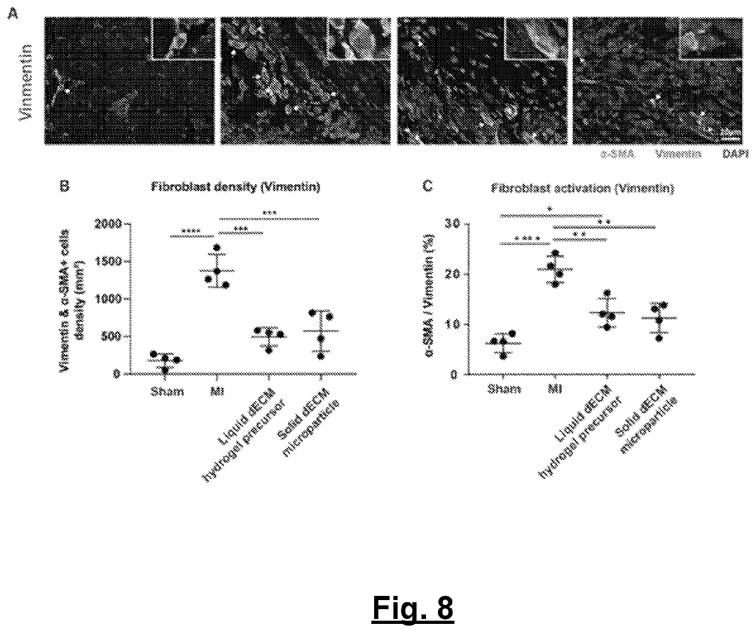

Heart fibrosis was examined by Masson's Trichrome staining at week 3 post-MI (FIG. 7A). Since no fibrotic tissue was observed in sham hearts, they were not included in the fibrosis analysis. The wall thickness in the infarct area was higher in dECM microparticles treated hearts than MI-control (FIG. 7B). Both dECM microparticles and dECM hydrogel precursor significantly lowered the fibrotic area in comparison to MI-control (FIG. 7C). Because fibroblasts differentiate to α-SMA⁺myofibroblasts and increase collagen deposition in the infarct area, we then evaluated fibroblast activation in the infarct and border zones. Cardiac fibroblasts are heterogeneous cell populations with no comprehensive cell marker. In this experiment, PDGFR-α (FIG. 7D) and Vimentin (FIG. 8A) were used to identify fibroblasts. In PDGFR-α positive cells, only dECM microparticles significantly reduced activated α-SMA⁺ fibroblasts density relative to MI-control (FIG. 7E), though a similar effect in fibroblast activation was observed in dECM microparticles and hydrogel precursor treated hearts (FIG. 7F). In Vimentin positive cells, fibroblast density was comparable for both dECM treatments (FIG. 8B); however, only dECM microparticles treated hearts lowered fibroblast activation to sham levels (FIG. 8C). The results demonstrate that dECM microparticles have high potency for lowering fibroblast activation and ventricular wall remodeling in post-MI hearts.

dECM Microparticles Promote Cardiomyocyte Cell Cycle Activity 3 Weeks Post-MI

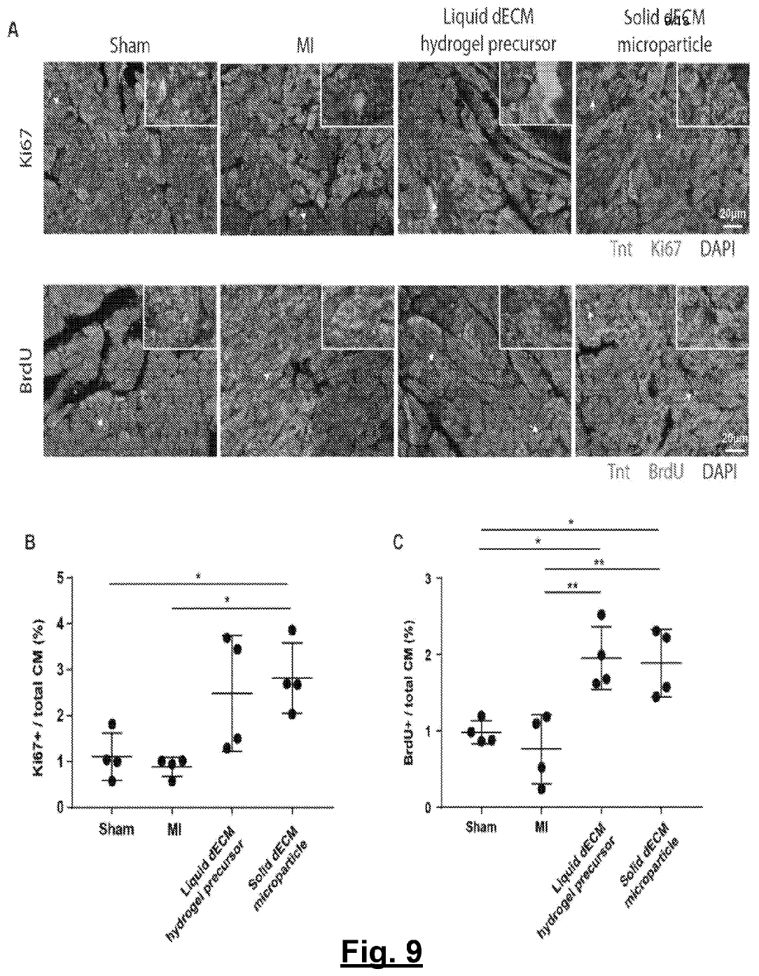

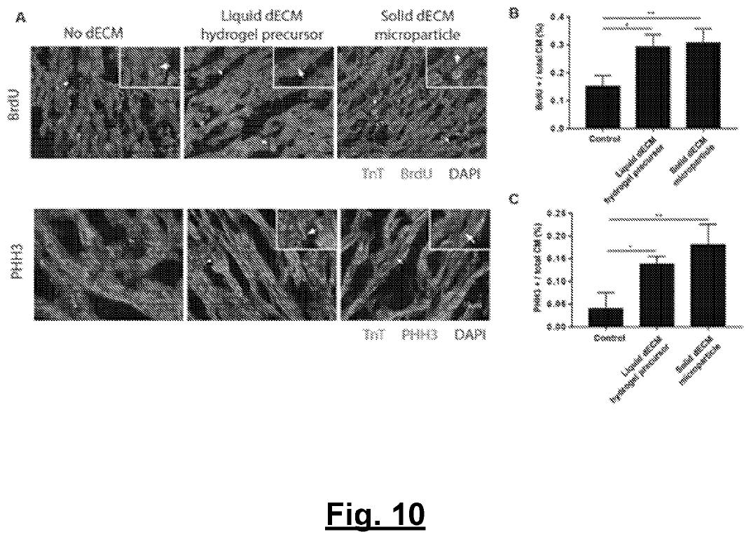

Measuring cardiomyocyte renewal in vivo is challenging because of the extremely low cardiomyocyte proliferation rate and bi-nucleation. Thus, cardiomyocyte cell cycle activity was used as an indicator of proliferation in this study. Cardiomyocyte cell cycle activity was examined by immunostaining for Ki67 protein and BrdU incorporation on week 3 post-MI (FIG. 9A). The number of Ki67⁺ cardiomyocytes is positively related to cardiomyocytes undergoing cytokinesis. The dECM microparticles, but not dECM hydrogel precursor, significantly increased Ki67⁺ cardiomyocytes compared to MI-control (FIG. 9B). BrdU incorporation in cardiomyocytes was significantly increased by dECM microparticles and dECM hydrogel precursor compared to sham and MI-control (FIG. 9C). A similar experiment was conducted using day 1 mice ventricle explant (FIG. 10A). Cardiomyocyte BrdU incorporation (FIG. 10B) and the frequency of PHH3⁺ cardiomyocytes (FIG. 10C) were increased significantly by both dECM microparticles and dECM hydrogel precursor treatments relative to the untreated control. Together, the results indicate that dECM microparticles robustly increase cardiomyocyte cell cycle activity in post-MI hearts.

dECM Microparticle Characterization of Stiffness, Protein Release, and Swelling

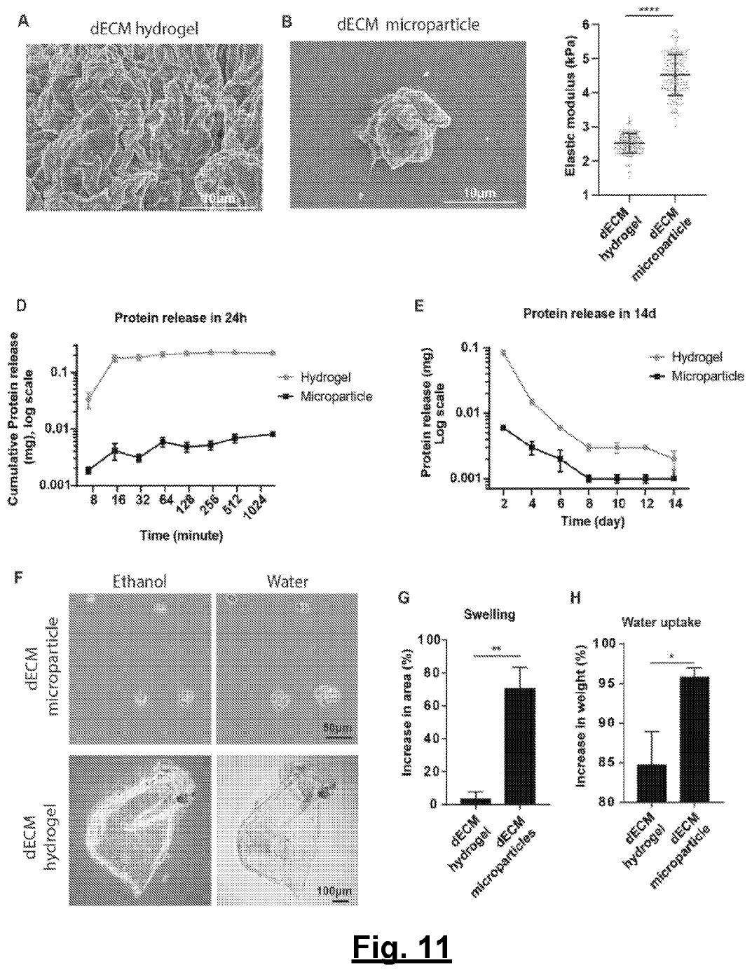

The dECM hydrogel was generated by gelation of liquid dECM hydrogel precursor at 37′C. SEM analysis showed similar topography for dECM hydrogel and microparticles (FIGS. 11A-B). Atomic force microscopy analysis indicated that dECM microparticle (4.5 kPa) has a significantly higher elastic modulus than hydrogel (2.5 kPa; p<0.001) (FIG. 11C).

The dECM microparticles and hydrogels were further characterized for protein release kinetics and swelling in PBS. No significant difference was observed in visual morphology after incubating in PBS for 14 days. However, a bulk release of proteins from dECM hydrogels occurred within 15 min after immersing in PBS buffer comprising most of the 24 h protein release (FIG. 11D). The dECM microparticles showed a repeatedly controlled release of proteins at a relatively constant rate over 24 h. In a 2-week study of protein release in infinite dilution, approximately 70% of total released proteins from dECM hydrogels were on day 2, and the released protein load from days 4 to 14 accounted for only 30% of the cumulative release (FIG. 11E) (see Table 2).

TABLE 2
Bulk Protein Release from dECM Hydrogels
and dECM Microparticles Over Time

| | |
|---|---|
| Hydrogel | Microparticle |

| | avg (mg) | std | avg (mg) | std |
|---|---|---|---|---|
| hours | | | | |
| 0 | 0.033289 | 0.010347 | 0.001851 | 0.000253 |
| 15 | 0.177718 | 0.024339 | 0.004138 | 0.001321 |
| 30 | 0.184556 | 0.021889 | 0.003082 | 0.000498 |
| 60 | 0.206871 | 0.022844 | 0.005873 | 0.001087 |
| 120 | 0.216392 | 0.020496 | 0.004811 | 0.000941 |
| 240 | 0.224557 | 0.019539 | 0.005165 | 0.000954 |
| 480 | 0.223866 | 0.016948 | 0.006821 | 0.001199 |
| 1440 | 0.221053 | 0.018104 | 0.008052 | 6.08E−05 |
| day | | | | |
| 2 | 0.083675 | 0.007186 | 0.005586 | 0.000457 |
| 4 | 0.014757 | 0.001451 | 0.003266 | 0.000671 |
| 6 | 0.005744 | 0.000158 | 0.001518 | 0.000724 |
| 8 | 0.0034 | 0.000342 | 0.001497 | 0.000119 |
| 10 | 0.002531 | 0.000523 | 0.001213 | 0.000143 |
| 12 | 0.003167 | 0.000134 | 0.00091 | 0.000149 |
| 14 | 0.001956 | 0.00063 | 0.001118 | 0.000366 |

In contrast, the dECM microparticles released 37% of total released proteins on day 2 and then decreased to approximately 10% from days 6 to 14. The mass of total proteins released from dECM hydrogels in 14 days was 6.7 times higher than dECM microparticles of the present disclosure.

To better understand the changes in dECM microparticles and hydrogels in aqueous buffer, swelling and water uptake were examined. The dECM microparticles showed observable changes in the area from dehydration to water exposure (FIG. 11F), while the dECM hydrogels did not show significant changes after immersion in water. We observed that dECM microparticles swelled by 70% in water, while dECM hydrogels swelled by 3% in water (FIG. 11G). Water uptake was examined by comparing dry sample weight to that after water absorption. The mass of dECM hydrogels increased by 85% after water absorption while that of dECM microparticles increased by 97% (FIG. 11H). While both dECM formulations have a high water capacity, the results indicate that dECM microparticles have a slower release of diffusible proteins.

dECM Microparticles Collagenase Digestion Resistance and Retention in Tissue

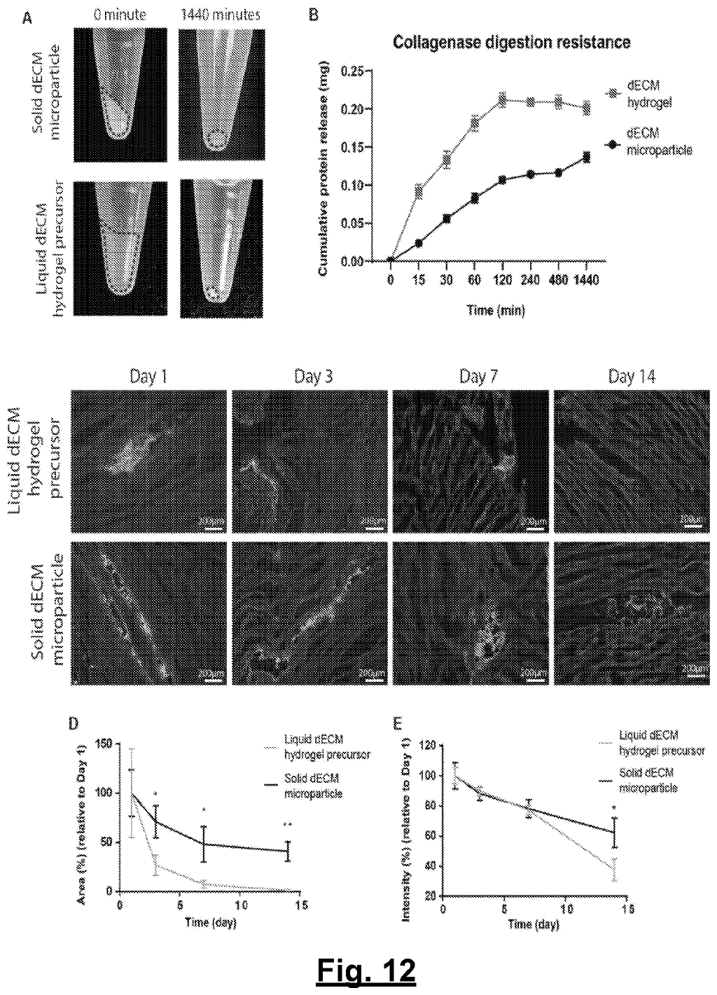

To determine if dECM microparticles are stable in a physiological environment, collagenase digestion resistance and sample retention were evaluated. A high concentration of collagenase (0.1 mg mL⁻¹) was used to accelerate the digestion. A larger mass of dECM microparticles was retained than hydrogels after 24 h digestion by visual inspection (FIG. 12A). The dECM hydrogels achieved the maximal cumulative protein release within 2 h of digestion. In contrast, dECM microparticles released proteins over the 24 h period (FIG. 12B). The retention of dECM was also examined by pre-labeling dECM with fluorescence markers. WGA-Alexa Fluor 488 labeled dECM microparticles and dECM hydrogel precursor were injected into explants from porcine hearts and examined at serial time points starting at day 1. Explants were incubated at 37° C. for 2 h before adding tissue culture media to induce dECM hydrogel precursor gelation. Samples were histologically prepared for fluorescence microscopy (FIG. 12C). The visible area of labeled dECM hydrogel precursor dropped rapidly in 7 days and was almost non-visible by day 14 (FIG. 12D). In contrast, dECM microparticles decreased by only 50% over 14 days. The fluorescence intensity of the fluorescent labels was also quantified. The dECM microparticles and dECM hydrogel precursor showed a similar decrement of sample fluorescence intensity from day 1 to day 7 (FIG. 12E). However, over the next 7 days, dECM microparticles exhibited a higher fluorescence intensity than dECM hydrogel precursor by day 14. These results suggest that dECM microparticles are more stable in natural tissue than liquid-derived dECM hydrogel.

dECM Microparticle Provides a Platform for Drug Delivery

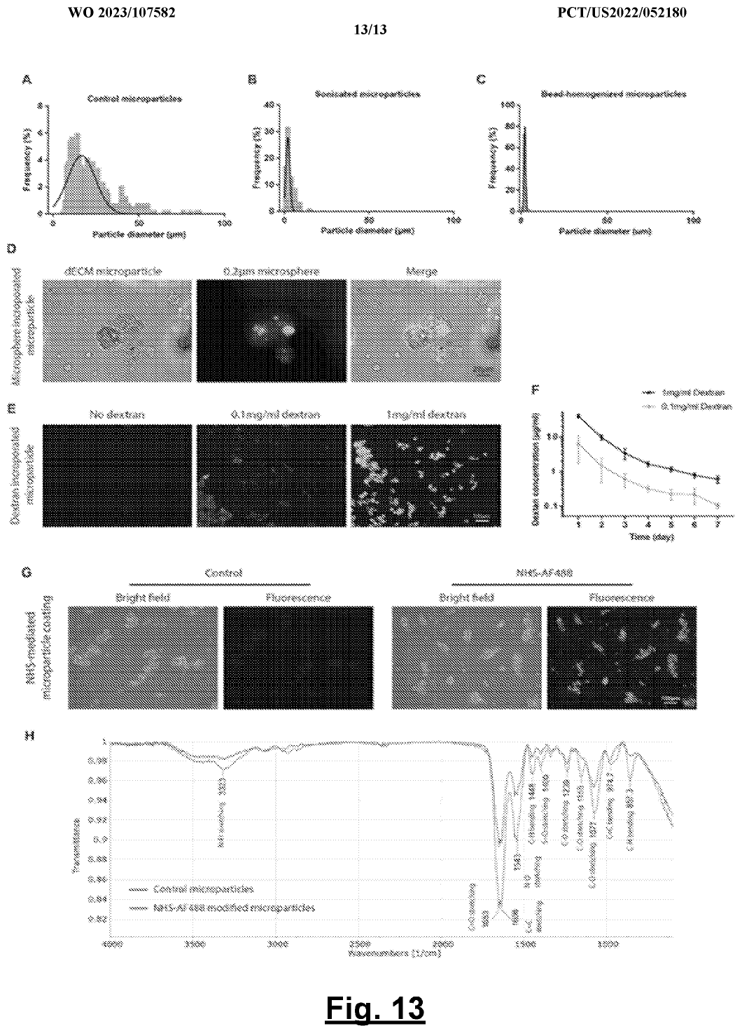

dECM microparticles offer potential as a platform for engineered drug delivery. The dECM microparticles have an average diameter of 22.8 μm (FIG. 13A) and could be lowered to 3.4 μm and 2.3 μm with increased monodispersity by post-electrospray sonication (FIG. 13B) and bead-homogenization (FIG. 13C), respectively. For proof-of-concept of using the particle as a vehicle for macromolecule-loaded polymers, polystyrene beads (200 nm in diameter) were incorporated into dECM microparticles by mixing solubilized dECM with the fluorescently labeled nanoparticles before continuing with the pre-established fabrication protocol (FIG. 13D). Nanoparticle incorporation could be titrated without any obvious impact on dECM microparticle size or aggregation. Similarly, to demonstrate direct small molecule loading, fluorescently-labeled dextran (10 kDa) was incorporated into dECM microparticles at two concentrations (0.1 and 1 mg mL⁻¹) (FIG. 13E). A controlled release of dextran was observed over 7 days by fluorescence measurements of supernatant by plate reader analysis (FIG. 13F). Finally, dECM microparticles were modified by chemical modification after electrospray for demonstration of altering the surface interface. NHS-AF488 was employed to determine if NHS-ester can conjugate molecules to dECM microparticles. An increased fluorescence was observed in NHS-AF488 functionalized dECM microparticles compared to the passively adsorbed control indicating AF488 molecules enrichment (FIG. 13G). FTIR analysis of NHS-AF488 functionalized dECM microparticles also indicated that NHS-AF488 was conjugated to microparticles by NHS-ester (FIG. 13H). The results suggest that dECM microparticles can be modified by directly loading drugs, incorporating drug-loaded nanoparticles, or surface coating and conjugation potentially for tissue targeting and drug delivery.

From the above description of the present disclosure, those skilled in the art will perceive improvements, changes and modifications. Such improvements, changes, and modifications are within the skill of those in the art and are intended to be covered by the appended claims. All patents, patent applications, and publications cited herein are incorporated by reference in their entirety.

1. A solid particle comprising decellularized cardiac tissue extracellular matrix.

2. The solid particle of claim 1 being a microparticle.

3. The solid particle of claim 2 having a diameter of about 1 μm to about 100 μm.

4. The solid particle of claim 3 having a diameter of about 20 μm.

5. The solid particle of claim 1 being spherical.

6. The solid particle of claim 1 wherein the cardiac tissue is human or porcine.

7. The solid particle of claim 1 wherein 100% of the particle is decellularized cardiac tissue extracellular matrix.

8. The solid particle of claim 1 not being a liquid, hydrogel, or sol-gel ex vivo.

9. The solid particle of claim 1 having an elastic modulus of about 1.5 kPa to about 6.5 kPa.

10. The solid particle of claim 9 having an elastic modulus greater than about 2.5 kPa, about 3 kPa, about 3.5 kPa, about 4 kPa, about 4.5 kPa, about 5 kPa, about 5.5 kPa, about 6 kPa, about 6.5 kPa, or greater than about 2.5 kPa but less than about 6.5 kPa.

11. The solid particle claim 10 having an elastic modulus of about 4.5 kPa.

12. The solid particle of claim 1, when exposed to or immersed in an aqueous environment, swell up to about 50-90% as compared to dECM particles or a hydrogel in a non-aqueous environment.

13. The solid particle of claim 12, when exposed to or immersed in an aqueous environment, swell up to about 70% as compared to dECM particles or a hydrogel in a non-aqueous environment.

14. The solid particle of claim 1 being resistant to complete enzymatic degradation about 1.5 times, about 2 times, about 2.5 times, about 3 times, about 3.5 times, about 4 times, about 4.5 times, about 5 times, about 5.5 times, about 6 times, about 6.5 times, about 7 times, about 7.5 times, about 8 times, about 8.5 times, about 9 times, about 9.5 times, about 10 times, about 10.5 times, or about 11 times greater than a time for complete enzymatic degradation of a hydrogel or hydrogel precursor.

15. The solid particle of claim 14 being resistant to complete enzymatic degradation for about 24 hours.

16. The solid particle of claim 1 wherein the solid particle, in a physiological environment, releases one or more biomolecules at a controlled release rate over a period of time.

17. The solid particle of claim 16 wherein the period of time is about 24 hours, about 24-28 hours, about 28-32 hours, about 32-36 hours, about 36-40 hours, about 40-44 hours, or about 44-48 hours.

18. The solid particle of claim 17 wherein the period of time is about 24 hours.

19. The solid particle of claim 1 wherein the solid particle, in a physiological environment, releases one or more biomolecules at a controlled release rate of about 0.5 ppm/hour to about 4 ppm/hour, or about 0.5-1 ppm/hour, or about 1-1.5 ppm/hour, or about 1.5-2 ppm/hour (e.g., 2 ppm/hour), or about 2-2.5 ppm/hour, or about 2.5-3 ppm/hour, or about 3-3.5 ppm/hour, or about 3.5-4 ppm/hour, or about 4-4.5 ppm/hour, or about 4.5-5 ppm/hour.

20. The solid particle of claim 19 wherein the solid particle, in a physiological environment, releases one or more biomolecules at a controlled release rate of about 2 ppm/hour.

21. The solid particle of claim 1 wherein the solid particle, in a physiological environment, releases one or more biomolecules at a controlled release rate of about 35 ppm over 24 hours to about 45 ppm over 24 hours, or about 35-36 ppm over 24 hours, or about 36-37 ppm over 24 hours, or about 37-38 ppm over 24 hours (e.g., about 38 ppm over 24 hours), or about 38-39 ppm over 24 hours, or about 39-40 ppm over 24 hours, or about 40-41 ppm over 24 hours, or about 41-42 ppm over 24 hours, or about 42-43 ppm over 24 hours, or about 43-44 ppm over 24 hours, or about 44-45 ppm over 24 hours.

22. The solid particle of claim 21 wherein the solid particle, in a physiological environment, releases one or more biomolecules at a controlled release rate of about 38 ppm over 24 hours.

23. The solid particle of claim 1 wherein no more than about 20-50% of total protein is released from the solid particle within about 20-50 hour(s) following contact of the solid particle with a physiological environment.

24. The solid particle of claim 23 wherein no more than about 20%, no more than about 25%, no more than about 30%, no more than about 35%, no more than about 40% (e.g., no more than about 37%), no more than about 45%, or no more than about 50% of total protein is released from the solid particle within about 20 hours, about 25 hours, about 30 hours, about 35 hours, about 40 hours, about 45 hours, or about 50 hours (e.g., about 48 hours) following contact of the solid particle with a physiological environment.

25. The solid particle of claim 1 wherein no more than about 37% of total protein is released from the solid particle within about 48 hours following contact of the solid particle with a physiological environment.

26. A solid particle comprising decellularized cardiac tissue extracellular matrix and at least one biomolecule that is chemically conjugated to the extracellular matrix.

27. The solid particle of claim 26 wherein the at least one biomolecule is a non-native biomolecule.

28. The solid particle of claim 27 wherein the biomolecule is a macromolecule or a small molecule.

29. The solid particle of claim 27 wherein the macromolecule is a macromolecule-loaded polymer.

30. The solid particle of claim 27 wherein the macromolecule or small molecule is one of a drug or a drug-loaded nanoparticle.

31. The solid particle of claim 26 wherein at least one surface of the solid particle is functionalized with one or more chemical and/or biological moieties.

32. The solid particle of claim 1 being formed by electrospraying.

33. The solid particle of claim 1 being formed by use of a microfluidic chip.

34. A composition comprising the solid particle of claim 1 and a second component.

35. A pharmaceutical composition comprising the solid particle of claim 1 and a pharmaceutically acceptable carrier.

36. A patch comprising a biodegradable bioscaffold seeded with a plurality of the solid particles of claim 1.

37. A solid particle consisting of decellularized cardiac tissue extracellular matrix.

38. A solid particle consisting of decellularized cardiac tissue extracellular matrix and at least one biomolecule chemically conjugated to the extracellular matrix.

39. A method of treating a cardiac disease or cardiac condition in a subject, comprising administering to the subject a therapeutically effective amount of the solid particles of claim 1, thereby treating the cardiac disease or cardiac condition.

40. The method of claim 39 wherein the subject has experienced a myocardial infarction prior to administration of the plurality of solid particles.

41. The method of claim 39 wherein, following administration of the plurality of solid particles, one or more of the following effects occurs in the subject: (1) preservation of cardiac function; (2) reduction in fibrosis and fibroblast activation; (3) stimulation of cardiomyocyte cell cycle activity; (4) angiogenesis in cardiac tissue; and (5) protection against ventricular wall remodeling.

42. The method of claim 39 wherein the plurality of solid particles is administered prior to, simultaneous with, or following treatment with a ventricular unloading modality.
